(19) United States
(12) Patent Application Publication
(10) Pub. No.: US 2020/0030330 A1
(43) Pub. Date: Jan. 30, 2020

(54) BRUTON'S TYROSINE KINASE INHIBITOR COMBINATIONS AND USES THEREOF

(71) Applicant: Pharmacyclics LLC, Sunnyvale, CA (US)

(72) Inventors: Danelle James, San Francisco, CA (US); Betty Y. Chang, Cupertino, CA (US); Taisei Kinoshita, San Jose, CA (US); Ssucheng J. Hsu, Pinole, CA (US); Jun Chen, San Jose, CA (US); Yujun Huang, San Diego, CA (US)

(21) Appl. No.: 16/289,511

(22) Filed: Feb. 28, 2019

Related U.S. Application Data
(62) Division of application No. 15/223,646, filed on Jul. 29, 2016, now abandoned.
(60) Provisional application No. 62/243,432, filed on Oct. 19, 2015.
(60) Provisional application No. 62/221,499, filed on Sep. 21, 2015.
(60) Provisional application No. 62/199,852, filed on Jul. 31, 2015.

(51) Int. Cl. A61K 31/519; A61K 45/06; A61P 35/00; A61K 31/436; A61K 31/506; A61K 31/337; C07K 16/28; A61K 39/395

(52) U.S. Cl. CPC A61K 31/519; A61K 45/06; A61P 35/00; A61K 31/436; C07K 2317/24; A61K 31/337; C07K 16/2863; A61K 39/3955; C07K 2317/76; A61K 31/506

(57) ABSTRACT

Disclosed arc compositions, methods, and kits for treating a solid tumor comprising co- administering to an individual in need thereof a BTK inhibitor and an mTOR inhibitor: a taxane, or an EGFR inhibitor.

### CROSS-REFERENCE TO RELATED APPLICATIONS

This application claims the benefit of U.S. Provisional Application Nos. 62/199,852, filed Jul. 31, 2015; 62/221,499, filed Sep. 21, 2015; and 62/243,432, filed Oct. 19, 2015; all of which are incorporated herein by reference in their entirety.

### BACKGROUND

Bruton's tyrosine kinase (BTK), a member of the Tec family of non-receptor tyrosine kinases, is a key signaling enzyme expressed in all hematopoietic cells types except T lymphocytes and natural killer cells. BTK plays an essential role in the B-cell signaling pathway linking cell surface B-cell receptor (BCR) stimulation to downstream intracellular responses.

### SUMMARY

In some embodiments, methods, compositions, kits, and reagents are provided herein for use in treating a solid tumor in a subject comprising administering to the subject a therapeutically effective amount of a combination comprising a BTK inhibitor and an mTOR inhibitor. In some embodiments, methods, compositions, kits, and reagents are provided herein for use in treating a solid tumor in a subject comprising administering to the subject a therapeutically effective amount of a combination comprising a BTK inhibitor and a pazopanib. In some embodiments, methods, compositions, kits, and reagents are provided herein for use in treating a solid tumor in a subject comprising administering to the subject a therapeutically effective amount of a combination comprising a BTK inhibitor and paclitaxel. In some embodiments, methods, compositions, kits, and reagents are provided herein for use in treating a solid tumor in a subject comprising administering to the subject a therapeutically effective amount of a combination comprising a BTK inhibitor and docetaxel. In some embodiments, methods, compositions, kits, and reagents are provided herein for use in treating a solid tumor in a subject comprising administering to the subject a therapeutically effective amount of a combination comprising a BTK inhibitor and an EGFR inhibitor.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a tumor volume comparison among treatments with ibrutinib, an mTOR inhibitor (sirolimus), and the combination thereof in a syngeneic renal cancer cell model (Renca).

FIG. 2 is a tumor volume comparison among treatments with ibrutinib, mTOR inhibitor everolimus, and the combination thereof in a xenograft renal cancer cell model (786-0).

FIG. 3 is a graphical representation of the combination of ibrutinib and an mTOR inhibitor (sirolimus) in a syngeneic renal cancer cell model (Renca).

FIG. 4 is a graphical representation of the combination of ibrutinib and an mTOR inhibitor (everolimus) in a xenograft renal cancer cell model (786-0).

FIG. 5A is a Western blot showing the effect of ibrutinib on the expression levels of various proteins in various renal cell carcinoma cell lines (A498, 769-P).

FIG. 5B is a Western blot showing the effect of ibrutinib on the expression levels of various proteins in various renal cell carcinoma cell lines (RENCA, ACHN).

FIG. 6A is a graphical representation of the effects of ibrutinib alone, mTOR inhibitor everolimus alone, and the combination of ibrutinib and everolimus on the relative cell growth of renal cancer cell line 769-P.

FIG. 6B is a graphical representation of the effects of ibrutinib alone, mTOR inhibitor everolimus alone, and the combination of ibrutinib and everolimus on the relative cell growth of renal cancer cell line ACHN.

FIG. 6C is a graphical representation of the effects of ibrutinib alone, mTOR inhibitor everolimus alone, and the combination of ibrutinib and everolimus on the relative cell growth of renal cancer cell line A498.

FIG. 7 is a Western blot showing the effect of ibrutinib, combined with mTOR inhibitor everolimus, on the expression levels of various proteins in the 769-P cell line.

FIG. 8 is a Western blot showing the effect of ibrutinib combined with mTOR inhibitor everolimus, on the expression levels of various proteins in the ACHN cell line.

FIG. 9A is a graphical representation of the effects of ibrutinib alone, pazopanib alone, and the combination of ibrutinib and pazopanib on cell growth inhibition in renal cancer cell line 769-P.

FIG. 9B is a graphical representation of the effects of ibrutinib alone, pazopanib alone, and the combination of ibrutinib and pazopanib on cell growth inhibition in renal cancer cell line ACHN.

FIG. 9C is a graphical representation of the effects of ibrutinib alone, pazopanib alone, and the combination of ibrutinib and pazopanib on cell growth inhibition in renal cancer cell line A498.

FIG. 10A is a graphical representation of the effect of the combination of ibrutinib and pazopanib on apoptosis in renal cancer cell line 769-P.

FIG. 10B is a graphical representation of the effect of the combination of ibrutinib and pazopanib on apoptosis in renal cancer cell line ACHN.

FIG. 10C is a graphical representation of the effect of the combination of ibrutinib and pazopanib on apoptosis in renal cancer cell line A498.

FIG. 11A is a Western blot showing the effect of ibrutinib, combined with pazopanib, on the expression levels of various proteins in the 769-P cell line.

FIG. 11B is a Western blot showing the effect of ibrutinib combined with pazopanib, on the expression levels of various proteins in the A498 cell line.

FIG. 11C is a Western blot showing the effect of ibrutinib combined with pazopanib, on the expression levels of various proteins in the ACHN cell line.

FIG. 12A is a graphical representation of the effects of vehicle, ibrutinib alone, mTOR inhibitor everolimus alone, or the combination of ibrutinib and everolimus, on tumor growth in 786-0 xenograft mouse model.

FIG. 12B is a graphical representation of the effects of vehicle, ibrutinib alone, mTOR inhibitor everolimus alone, or the combination of ibrutinib and everolimus, on tumor growth in RENCA syngeneic mouse model.

FIG. 13 is a graphical representation of the effects of vehicle, ibrutinib alone, cetuximab alone, or the combination of ibrutinib and cetuximab in FaDu human head and neck xenografts.

FIG. 14 is a graphical representation of the effects of vehicle, ibrutinib alone, everolimus alone, CGI-1746 alone, or the combination of ibrutinib or CGI-1746 with everolimus in a xenograft renal cancer cell model (786-0).

### DETAILED DESCRIPTION

In some embodiments, methods for treating a solid tumor are provided. The method includes the step of co-administering to an individual in need thereof a BTK inhibitor and an mTOR inhibitor. In some embodiments, the combination provides a synergistic effect compared to administration of the Btk inhibitor or the mTOR inhibitor alone. In some embodiments, the BTK inhibitor is ibrutinib. Exemplary mTOR inhibitors are everolimus and sirolimus. In some embodiments, the solid tumor is a carcinoma. Exemplary solid tumors include breast cancer, pancreatic cancer, colorectal cancer, bladder cancer, lung cancer, non-small cell lung cancer, large cell lung cancer, prostate cancer, ovarian cancer, bile duct cancer, renal cell carcinoma, and kidney cancer. In some embodiments, the solid tumor is renal cell carcinoma. In some embodiments, the solid tumor is kidney cancer. In some embodiments, the solid tumor is a relapsed or refractory solid tumor. In some embodiments, the solid tumor is a treatment naïve solid tumor. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered at a dosage of about 420 mg/day. In some embodiments, ibrutinib is administered at a dosage of about 560 mg/day. In some embodiments, ibrutinib is administered at a dosage of about 700 mg/day. In some embodiments, ibrutinib is administered at a dosage of about 840 mg/day. In some embodiments, ibrutinib is administered orally. In some embodiments, ibrutinib and the mTOR inhibitor are administered simultaneously, sequentially, or intermittently.

In some embodiments, methods for treating a renal cell carcinoma are provided. The method includes the step of co-administering to an individual in need thereof a BTK inhibitor and an mTOR inhibitor. In some embodiments, the combination provides a synergistic effect compared to administration of the BTK inhibitor or the mTOR inhibitor alone. In some embodiments, the Btk inhibitor is ibrutinib. Exemplary mTOR inhibitors are everolimus and sirolimus. In some embodiments, the renal cell carcinoma is relapsed or refractory. In some embodiments, the renal cell carcinoma is treatment naïve. In some embodiments, the subject has had at least one prior therapy. In some embodiments, the prior therapy comprises administration of a vascular endothelial growth factor inhibitor (VEGF-TKI).

In some embodiments, a pharmaceutical composition is provided. The pharmaceutical composition includes a BTK inhibitor, an mTOR inhibitor (e.g., everolimus or sirolimus), and a pharmaceutically-acceptable excipient. In some embodiments, the BTK inhibitor is ibrutinib. In some embodiments, the combination is in a combined dosage form. In some embodiments, the combination is in separate dosage forms.

In some embodiments, methods for treating a solid tumor are provided. The method includes the step of co-administering to an individual in need thereof a BTK inhibitor and pazopanib or a salt thereof. In some embodiments, the combination provides a synergistic effect compared to administration of the BTK inhibitor or pazopanib or salt thereof alone. In some embodiments, the BTK inhibitor is ibrutinib. In some embodiments, the solid tumor is a carcinoma. Exemplary solid tumors include breast cancer, pancreatic cancer, colorectal cancer, bladder cancer, lung cancer, non-small cell lung cancer, large cell lung cancer, prostate cancer, ovarian cancer, bile duct cancer, renal cell carcinoma, and kidney cancer. In some embodiments, the solid tumor is renal cell carcinoma. In some embodiments, the solid tumor is kidney cancer. In some embodiments, the solid tumor is a relapsed or refractory solid tumor. In some embodiments, the solid tumor is a treatment naïve solid tumor. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered at a dosage of about 420 mg/day. In some embodiments, ibrutinib is administered at a dosage of about 560 mg/day. In some embodiments, ibrutinib is administered at a dosage of about 700 mg/day. In some embodiments, ibrutinib is administered at a dosage of about 840 mg/day. In some embodiments, ibrutinib is administered orally. In some embodiments, ibrutinib and pazopanib or salt thereof are administered simultaneously, sequentially, or intermittently.

In some embodiments, a pharmaceutical composition is provided. The pharmaceutical composition includes a BTK inhibitor, pazopanib, and a pharmaceutically-acceptable excipient. In some embodiments, the BTK inhibitor is ibrutinib. In some embodiments, the combination is in a combined dosage form. In some embodiments, the combination is in separate dosage forms.

In some embodiments, methods of treating a solid tumor are provided. The method includes the step of co-administering to an individual in need thereof a combination of a BTK inhibitor and paclitaxel. In some embodiments, the combination provides a synergistic effect compared to the administration of the BTK inhibitor or paclitaxel alone. In some embodiments, the BTK inhibitor is ibrutinib. In some embodiments, the solid tumor is a carcinoma. In some embodiments, the carcinoma is an urotheilial carcinoma. In some embodiments, the solid tumor is a relapsed or refractory solid tumor. In some embodiments, the solid tumor is a treatment naïve solid tumor. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered at a dosage of about 420 mg/day. In some embodiments, ibrutinib is administered at a dosage of about 560 mg/day. In some embodiments, ibrutinib is administered at a dosage of about 700 mg/day. In some embodiments, ibrutinib is administered at a dosage of about 840 mg/day. In some embodiments, paclitaxel is administered once per week. In some embodiments, paclitaxel is administered at a dosage of about 80 mg/m² once per week. In some embodiments, ibrutinib and paclitaxel are administered simultaneously, sequentially, or intermittently. In some embodiments, the subject has had at least one prior therapy. In some embodiments, the prior therapy does not include administration of a taxane.

In some embodiments, a dosing regimen for the treatment of an urothelial carcinoma in a subject in need thereof is provided. The dosing regimen includes administering to the subject a combination comprising ibrutinib and paclitaxel, wherein ibrutinib and paclitaxel are administered concurrently in at least one cycle. In some embodiments, each cycle is 21 days. In some embodiments, paclitaxel is administered once per week. In some embodiments, ibrutinib is administered at a dosage of about 40 mg/day to about 1000 mg/day. In some embodiments, ibrutinib is administered at a dosage of about 420 mg/day. In some embodiments, ibrutinib is administered at a dosage of about 560 mg/day. In some embodiments, ibrutinib is administered at a dosage of about 700 mg/day. In some embodiments, ibrutinib is administered at a dosage of about 840 mg/day. In some embodiments, paclitaxel is administered at a dosage of about 80 mg/m² once per week. In some embodiments, the subject has had at least one prior therapy. In some embodiments, the prior therapy does not include administration of a taxane.

In some embodiments, a pharmaceutical composition is provided. The pharmaceutical composition includes a BTK inhibitor, paclitaxel, and a pharmaceutically-acceptable excipient. In some embodiments, the BTK inhibitor is ibrutinib. In some embodiments, the combination is in a combined dosage form. In some embodiments, the combination is in separate dosage forms.

In some embodiments, methods of treating a solid tumor are provided. The method includes the step of co-administering to an individual in need thereof a combination of a BTK inhibitor and docetaxel. In some embodiments, the combination provides a synergistic effect compared to the administration of the BTK inhibitor or docetaxel alone. In some embodiments, the BTK inhibitor is ibrutinib. In some embodiments, the solid tumor is an adenocarcinoma. In some embodiments, the adenocarcinoma is a gastric adenocarcinoma. In some embodiments, the solid tumor is a relapsed or refractory solid tumor. In some embodiments, the solid tumor is a treatment naïve solid tumor. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered at a dosage of about 420 mg/day. In some embodiments, ibrutinib is administered at a dosage of about 560 mg/day. In some embodiments, ibrutinib is administered at a dosage of about 700 mg/day. In some embodiments, ibrutinib is administered at a dosage of about 840 mg/day. In some embodiments, docetaxel is administered once every three weeks. In some embodiments, docetaxel is administered at a dosage of about 75 mg/m² once every three weeks. In some embodiments, ibrutinib and docetaxel are administered simultaneously, sequentially, or intermittently. In some embodiments, the subject has had at least one prior therapy. In some embodiments, the prior therapy does not include administration of a taxane. In some embodiments the prior therapy comprises a fluoropyrimidine (5-FU) based regimen.

In some embodiments, a dosing regimen for the treatment of a gastric adenocarcinoma in a subject in need thereof is provided. The dosing regimen includes administering to the subject a combination comprising ibrutinib and docetaxel, wherein ibrutinib and docetaxel are administered concurrently in at least one cycle. In some embodiments, each cycle is 21 days. In some embodiments, docetaxel is administered once every three weeks. In some embodiments, ibrutinib is administered at a dosage of about 40 mg/day to about 1000 mg/day. In some embodiments, ibrutinib is administered at a dosage of about 420 mg/day. In some embodiments, ibrutinib is administered at a dosage of about 560 mg/day. In some embodiments, ibrutinib is administered at a dosage of about 700 mg/day. In some embodiments, ibrutinib is administered at a dosage of about 840 mg/day. In some embodiments, docetaxel is administered at a dosage of about 75 mg/m² once every three weeks. In some embodiments, docetaxel is administered at least once every cycle. In some embodiments, the subject has had at least one prior therapy. In some embodiments, the prior therapy does not include administration of a taxane. In some embodiments the prior therapy comprises a fluoropyrimidine (5-FU) based regimen.

In some embodiments, a pharmaceutical composition is provided. The pharmaceutical composition includes a BTK inhibitor, docetaxel, and a pharmaceutically-acceptable excipient. In some embodiments, the BTK inhibitor is ibrutinib. In some embodiments, the combination is in a combined dosage form. In some embodiments, the combination is in separate dosage forms.

In some embodiments, methods of treating a solid tumor are provided. The method includes the step of co-administering to an individual in need thereof a combination of a BTK inhibitor and an EGFR inhibitor. In some embodiments, the combination provides a synergistic effect compared to the administration of the BTK inhibitor or the EGFR inhibitor alone. In some embodiments, the BTK inhibitor is ibrutinib. In some embodiments, the EGFR inhibitor is cetuximab. In some embodiments, the solid tumor is an adenocarcinoma. In some embodiments, the adenocarcinoma is colorectal cancer. In some embodiments, the solid tumor is a head and neck cancer. In some embodiments, the solid tumor is a relapsed or refractory solid tumor. In some embodiments, the solid tumor is a treatment naïve solid tumor. In some embodiments, ibrutinib is administered once a day, two times per day, three times per day, four times per day, or five times per day. In some embodiments, ibrutinib is administered at a dosage of about 40 mg/day to about 1000 mg/day. In some embodiments, ibrutinib is administered at a dosage of about 420 mg/day. In some embodiments, ibrutinib is administered at a dosage of about 560 mg/day. In some embodiments, ibrutinib is administered at a dosage of about 700 mg/day. In some embodiments, ibrutinib is administered at a dosage of about 840 mg/day. In some embodiments, the combination of ibrutinib and cetuximab is administered concurrently in at least one cycle. In some embodiments, each cycle is 21 days. In some embodiments, cetuximab is administered at a first dosage and a second dosage, wherein the first dosage is the initial dosage of cetuximab and the second dosage is each subsequent dosage. In some embodiments, the first dosage is about 400 mg/m². In some embodiments, the second dosage is administered weekly. In some embodiments, the second dosage is about 250 mg/m². In some embodiments, ibrutinib and cetuximab are administered simultaneously, sequentially, or intermittently. In some embodiments, the subject has had at least one prior therapy. In some embodiments, the prior therapy comprises both an irinotecan-based regimen and an oxaliplatin-based regimen. In some embodiments, the prior therapy comprises an oxaliplatin-based regimen. In some embodiments, the prior therapy comprises an irinotecan-based regimen. In some embodiments, the subject is considered intolerant to irinotecan.

In some embodiments, a dosing regimen for the treatment of a colorectal cancer in a subject in need thereof is provided. The dosing regimen includes administering to the subject a combination comprising ibrutinib and cetuximab, wherein ibrutinib and cetuximab are administered concurrently in at least one cycle. In some embodiments, each cycle is 21 days. In some embodiments, cetuximab is administered at a first dosage and a second dosage, wherein the first dosage is the initial dosage of cetuximab and the second dosage is each subsequent dosage. In some embodiments, the first dosage is about 400 mg/m². In some embodiments, the second dosage is administered weekly. In some embodiments, the second dosage is about 250 mg/m². In some embodiments, ibrutinib is administered at a dosage of about 40 mg/day to about 1000 mg/day. In some embodiments, ibrutinib is administered at a dosage of about 420 mg/day. In some embodiments, ibrutinib is administered at a dosage of about 560 mg/day. In some embodiments, ibrutinib is administered at a dosage of about 700 mg/day. In some embodiments, ibrutinib is administered at a dosage of about 840 mg/day. In some embodiments, the subject has had at least one prior therapy. In some embodiments, the prior therapy comprises both an irinotecan-based regimen and an oxaliplatin-based regimen. In some embodiments, the prior therapy comprises an oxaliplatin-based regimen. In some embodiments, the prior therapy comprises an irinotecan-based regimen. In some embodiments, the subject is considered intolerant to irinotecan.

In some embodiments, a pharmaceutical composition is provided. The pharmaceutical composition includes a BTK inhibitor, cetuximab, and a pharmaceutically-acceptable excipient. In some embodiments, the BTK inhibitor is ibrutinib. In some embodiments, the combination is in a combined dosage form. In some embodiments, the combination is in separate dosage forms.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, “anti-cancer agent” refers to mTOR inhibitor(s); pazopanib or a salt thereof; paclitaxel; docetaxel; and/or EGFR inhibitors (e.g., cetuximab). As used herein, “anticancer agent” can also be used to refer to a third agent as disclosed herein.

“Antibodies” and “immunoglobulins” (Igs) are glycoproteins having the same structural characteristics. The terms are used synonymously. In some instances the antigen specificity of the immunoglobulin may be known.

The term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

The term “urothelial carcimona” may refer to a transitional cell carcinoma; kidney cancer; bladder cancer; ureter cancer; cancer of the renal pelvis; cancer of the cells lining the urinary tract; transitional cell carcinoma of the renal pelvis; transitional cell carcinoma of the ureter; transitional cell carcinoma of the bladder; transitional cell carcinoma of the urethra.

The term “taxane” as used herein includes paclitaxel and docetaxel.

The suffix “ene” appended to a group indicates that such a group is a diradical. By way of example only, a methylene is a diradical of a methyl group, that is, it is a —CH₂— group; and an ethylene is a diradical of an ethyl group, i.e., —CH₂CH₂—.

As used herein, C₁-Cₓ includes C₁-C₂, C₁-C₃ . . . C₁-Cₓ, i.e., one to two carbon atoms, one to three carbon atoms . . . one to x carbon atoms.

An “alkyl” group refers to a saturated, branched or straight chain hydrocarbon group. The “alkyl” moiety optionally has 1 to 10 carbon atoms (whenever it appears herein, a numerical range such as “1 to 10” refers to each integer in the given range; e.g., “1 to 10 carbon atoms” means that the alkyl group is selected from a moiety having 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups are optionally substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. “Lower alkyl” having 1 to 6 carbon atoms.

The term “alkenyl” refers to a hydrocarbon group containing at least one double bond formed by two carbon atoms that is not part of an aromatic group. An example of an alkenyl group is —C(R)═C(R)—R, wherein R refers to the remaining portions of the alkenyl group, which are either the same or different. The alkenyl moiety is optionally branched, straight chain, or cyclic (in which case, it is also known as a “cycloalkenyl” group). Depending on the structure, an alkenyl group includes a monoradical or a diradical (i.e., an alkenylene group). Alkenyl groups are optionally substituted. Non-limiting examples of an alkenyl group include —CH═CH₂, —C(CH₃)═CH₂, —CH═CHCH₃, —C(CH₃)═CHCH₃. Alkenylene groups include, but are not limited to, —CH═CH—, —C(CH₃)═CH—, —CH═CHCH₂—, —CH═CHCH₂CH₂— and —C(CH₃)═CHCH₂—. Alkenyl groups optionally have 2 to 10 carbons, and if a “lower alkenyl” having 2 to 6 carbon atoms.

The term “alkynyl” refers to a branched or straight chain hydrocarbon group containing at least one triple bond formed by two carbon atoms. An example of an alkynyl group is —C≡C—R, wherein R refers to the remaining portions of the alkynyl group, which is either the same or different. The “R” portion of the alkynyl moiety may be branched, straight chain, or cyclic. Depending on the structure, an alkynyl group includes a monoradical or a diradical (i.e., an alkynylene group). Alkynyl groups are optionally substituted. Non-limiting examples of an alkynyl group include, but are not limited to, —C≡CH, —C≡CCH₃, —C≡CCH₂CH₃, —C≡—, and —C≡CCH₂—. Alkynyl groups optionally have 2 to 10 carbons, and if a “lower alkynyl” having 2 to 6 carbon atoms.

An “alkoxy” group refers to an (alkyl)O— group, where alkyl is as defined herein.

An “amide” is a chemical moiety with the formula —C(O)NHR or —NHC(O)R, where R is selected from alkyl, cycloalkyl, aryl, heteroaryl (bonded through a ring carbon) and heteroalicyclic (bonded through a ring carbon). In some embodiments, an amide moiety forms a linkage between an amino acid or a peptide molecule and a compound described herein, thereby forming a prodrug. Any amine, or carboxyl side chain on the compounds described herein can be amidified. The procedures and specific groups to make such amides are found in sources such as Greene and Wuts, Protective Groups in Organic Synthesis, 3^{rd} Ed., John Wiley & Sons, New York, N.Y., 1999, which is incorporated herein by reference for this disclosure.

The term “ester” refers to a chemical moiety with formula —COOR, where R is selected from alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are found in sources such as Greene and Wuts, Protective Groups in Organic Synthesis, 3^{rd} Ed., John Wiley & Sons, New York, N.Y., 1999, which is incorporated herein by reference for this disclosure.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic radical that contains only carbon and hydrogen, and is optionally saturated, or partially unsaturated. Cycloalkyl groups include groups having from 3 to 10 ring atoms. Illustrative examples of cycloalkyl groups include the following moieties:

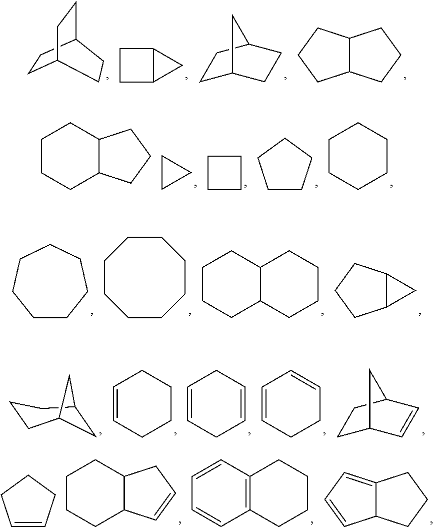

and the like. Depending on the structure, a cycloalkyl group is either a monoradical or a diradical (e.g., a cycloalkylene group), and if a “lower cycloalkyl” having 3 to 8 carbon atoms.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6-membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic (heteroaryl) groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, are optionally C-attached or N-attached where such is possible. For instance, a group derived from pyrrole includes pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The terms “heteroaryl” or, alternatively, “heteroaromatic” refers to an aromatic group that includes one or more, such as one to four, ring heteroatoms selected from nitrogen, oxygen and sulfur. Heteroaryl rings can be formed by five, six, seven, eight, nine, or more than nine, e.g., up to fourteen, ring atoms. An N-containing “heteroaromatic” or “heteroaryl” moiety refers to an aromatic group in which at least one of the skeletal atoms of the ring is a nitrogen atom. Illustrative examples of heteroaryl groups include the following moieties:

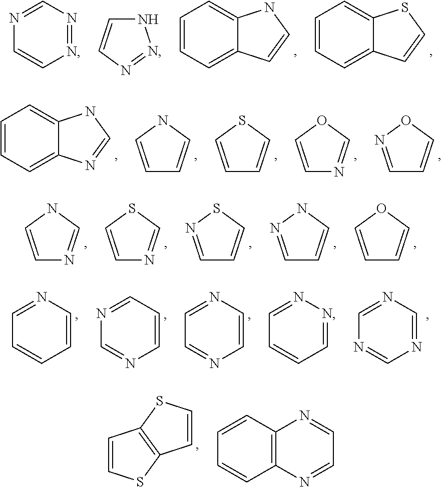

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more, such as one to four, atoms forming the ring are a heteroatom. A “non-aromatic heterocycle” or “heterocycloalkyl” group refers to a cycloalkyl group that includes at least one heteroatom selected from nitrogen, oxygen and sulfur. In some embodiments, the radicals are fused with an aryl or heteroaryl. Heterocycloalkyl rings can be formed by three, four, five, six, seven, eight, nine, or more than nine, e.g., up to fourteen, ring atoms. Heterocycloalkyl rings can be optionally substituted. In certain embodiments, non-aromatic heterocycles contain one or more carbonyl (═O) or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

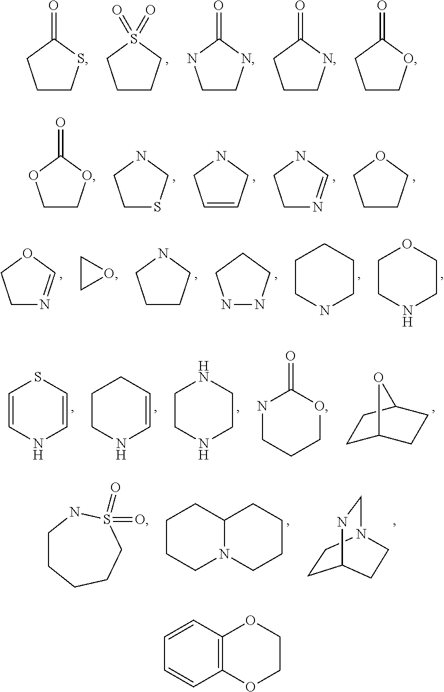

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

The term “halo” or, alternatively, “halogen” or “halide” means fluoro, chloro, bromo and iodo.

The term “haloalkyl,” refers to alkyl structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

The term “fluoroalkyl,” as used herein, refers to alkyl group in which at least one hydrogen is replaced with a fluorine atom. Examples of fluoroalkyl groups include, but are not limited to, —CF₃, —CH₂CF₃, —CF₂CF₃, —CH₂CH₂CF₃ and the like.

As used herein, the term “heteroalkyl” refers to optionally substituted alkyl radicals in which one or more, such as one to three or one to two, skeletal chain atoms is a heteroatom, e.g., oxygen, nitrogen, sulfur, silicon, phosphorus or combinations thereof. The heteroatom(s) are placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, in some embodiments, up to two heteroatoms are consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond” or “single bond” refers to a chemical bond between two atoms, or two moieties when the atoms joined by the bond are considered to be part of larger sub structure.

The term “moiety” refers to a specific segment or functional group of a molecule. Chemical moieties are often recognized chemical entities embedded in or appended to a molecule.

The term “optionally substituted” or “substituted” means that the referenced group may be substituted with one or more additional group(s), by way of example, individually and independently selected from cyano, halo, acyl, nitro, haloalkyl, fluoroalkyl, amino, including mono- and di-substituted amino groups, and the protected derivatives thereof, or L^{s}R^{s}, wherein each L^{s} is independently selected from a bond, —O—, —C(═O)—, —S—, —S(═O)—, —S(═O)₂—, —NH—, —NR^{s}—, —NHC(O)—, —C(O)NH—, —S(═O)₂NH—, —NHS(═O)₂—, —OC(O)NH—, —NHC(O)O—, -(substituted or unsubstituted C₁-C₆ alkylene), or -(substituted or unsubstituted C₂-C₆ alkenylene); and each R^{s} is independently selected from H, (substituted or unsubstituted C₁-C₄alkyl), (substituted or unsubstituted C₃-C₆cycloalkyl), (substituted or unsubstituted heterocycloalkyl), (substituted or unsubstituted aryl), (substituted or unsubstituted heteroaryl), or (substituted or unsubstituted heteroalkyl). The protecting groups that form the protective derivatives of the above substituents include those found in sources such as Greene and Wuts, above.

### Solid Tumors

In some embodiments, the composition is for use in treatment of a solid tumor. In some embodiments, the composition is for use in treatment of a sarcoma or carcinoma. In some embodiments, the composition is for use in treatment of a sarcoma. In some embodiments, the composition is for use in treatment of a carcinoma. In some embodiments, the carcinoma is renal cell carcinoma. In some embodiments, the carcinoma is urothelial carcinoma. In some embodiments, the carcinoma is transitional cell carcinoma. In some embodiments, the carcinoma is a carcinoma of the bladder, ureters, and/or renal pelvis. In some embodiments, the carcinoma is renal transitional cell carcinoma or renal urothelial carcinoma. In some embodiments, the carcinoma is of the kidney, urinary tract, ureter, urethra, and/or urachus. In some embodiments, the carcinoma is of the prostate. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extrasketetal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epithelioid cell differentiation; osteosarcoma; parosteal osteosarcoma; neoplasm with perivascular epithelioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from an adenocarcinoma, squamous cell carcinoma, adenosquamous carcinoma, anaplastic carcinoma, large cell carcinoma, or small cell carcinoma. In some embodiments, the carcinoma is selected from kidney cancer, anal cancer; appendix cancer; bile duct cancer (i.e., cholangiocarcinoma); bladder cancer; brain tumor; breast cancer; cervical cancer; colon cancer; cancer of Unknown Primary (CUP); esophageal cancer; eye cancer; fallopian tube cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the carcinoma is breast cancer. In some embodiments, the breast cancer is invasive ductal carcinoma, ductal carcinoma in situ, invasive lobular carcinoma, or lobular carcinoma in situ. In some embodiments, the carcinoma is pancreatic cancer. In some embodiments, the pancreatic cancer is adenocarcinoma, or islet cell carcinoma. In some embodiments, the carcinoma is colorectal cancer. In some embodiments, the colorectal cancer is adenocarcinoma. In some embodiments, the solid tumor is a colon polyp. In some embodiments, the colon polyp is associated with familial adenomatous polyposis. In some embodiments, the carcinoma is bladder cancer. In some embodiments, the bladder cancer is transitional cell bladder cancer, squamous cell bladder cancer, or adenocarcinoma. In some embodiments, the carcinoma is lung cancer. In some embodiments, the lung cancer is a non-small cell lung cancer. In some embodiments, the non-small cell lung cancer is adenocarcinoma, squamous-cell lung carcinoma, or large-cell lung carcinoma. In some embodiments, the non-small cell lung cancer is large cell lung cancer. In some embodiments, the lung cancer is a small cell lung cancer. In some embodiments, the carcinoma is prostate cancer. In some embodiments, the prostate cancer is adenocarcinoma or small cell carcinoma. In some embodiments, the carcinoma is ovarian cancer. In some embodiments, the ovarian cancer is epithelial ovarian cancer. In some embodiments, the carcinoma is bile duct cancer. In some embodiments, the bile duct cancer is proximal bile duct carcinoma or distal bile duct carcinoma. In some embodiments, the cancer is gastric cancer or stomach cancer. In some embodiments, the gastric cancer is a carcinoma. In some embodiments, the gastric cancer is gastric adenocarcinoma. In some embodiments, the cancer is a cancer of the esophagogastric junction (GEJ). In some embodiments, the cancer is an adenocarcinoma of the esophagogastric junction (GEJ). In some embodiments, the cancer is a carcimona of the esophagogastric junction (GEJ). In some embodiments, the cancer is an esophageal cancer. In some embodiments, the cancer is head and neck cancer.

In some embodiments, the solid tumor is a relapsed and/or refractory solid tumor. In some embodiments, the solid tumor is a relapsed and/or refractory urothelial carcinoma. In some embodiments, the solid tumor is a relapsed and/or refractory gastric adenocarcinoma. In some embodiments, the solid tumor is a relapsed and/or refractory colorectal carcinoma. In some embodiments, the solid tumor is a relapsed and/or refractory colorectal cancer. In some embodiments, the gastric cancer is a relapsed and/or refractory carcinoma. In some embodiments, the gastric cancer is a relapsed and/or refractory gastric adenocarcinoma. In some embodiments, the cancer is a relapsed and/or refractory cancer of the esophagogastric junction (GEJ). In some embodiments, the cancer is a relapsed and/or refractory adenocarcinoma of the esophagogastric junction (GEJ). In some embodiments, the cancer is a relapsed and/or refractory carcimona of the esophagogastric junction (GEJ). In some embodiments, the cancer is a relapsed and/or refractory esophageal cancer.

In some embodiments, the solid tumor is a treatment naïve solid tumor. In some embodiments, the solid tumor is a treatment naïve urothelial carcinoma. In some embodiments, the solid tumor is treatment naive gastric adenocarcinoma. In some embodiments, the solid tumor is treatment naïve colorectal carcinoma. In some embodiments, the solid tumor is a treatment naive colorectal cancer. In some embodiments, the gastric cancer is a treatment naive carcinoma. In some embodiments, the gastric cancer is a treatment naïve gastric adenocarcinoma. In some embodiments, the cancer is a treatment naive cancer of the esophagogastric junction (GEJ). In some embodiments, the cancer is a treatment naive adenocarcinoma of the esophagogastric junction (GEJ). In some embodiments, the cancer is a treatment naive carcimona of the esophagogastric junction (GEJ). In some embodiments, the cancer is a treatment naive esophageal cancer.

In some embodiments, the solid tumor is a metastatic and/or advanced solid tumor. In some embodiments, the solid tumor is a metastatic renal cell carcinoma. In some embodiments, the solid tumor is a metastatic and/or advanced urothelial carcinoma. In some embodiments, the solid tumor is a metastatic and/or advanced gastric adenocarcinoma. In some embodiments, the solid tumor is a metastatic and/or advanced colorectal carcinoma. In some embodiments, the solid tumor is a metastatic colorectal adenocarcinoma. In some embodiments, the solid tumor is a metastatic and/or advanced colorectal cancer. In some embodiments, the gastric cancer is a metastatic and/or advanced carcinoma. In some embodiments, the gastric cancer is a metastatic and/or advanced gastric adenocarcinoma. In some embodiments, the cancer is a metastatic and/or advanced cancer of the esophagogastric junction (GEJ). In some embodiments, the cancer is a metastatic and/or advanced adenocarcinoma of the esophagogastric junction (GEJ). In some embodiments, the cancer is a metastatic and/or advanced carcimona of the esophagogastric junction (GEJ). In some embodiments, the cancer is a metastatic and/or advanced esophageal cancer.

In some embodiments, the solid tumor is not characterized by an over-expression of an ABC transporter. In some embodiments, the solid tumor is not characterized by an over-expression of ABC-transporters such as, but not limited to, ATP-binding cassette subfamily B member 1 (ABCB1), ATP-binding cassette sub-family G member 2 (ABCG2), ATP-binding cassette sub-family C member 1 (ABCC1), ATP-binding cassette sub-family C member 2 (ABCC2), or ATP-binding cassette sub-family C member 10 (ABCC10).

In some embodiments, the solid tumor is not breast cancer. In some embodiments, the solid tumor is not prostate cancer. In some embodiments, the solid tumor is not pancreatic cancer. In some embodiments, the solid tumor is not lung cancer.

In some embodiments, the solid tumor is not resistant to paclitaxel. In some embodiments, the solid tumor is resistant to paclitaxel. In some embodiments, the solid tumor is not resistant to ibrutinib. In some embodiments, the solid tumor is resistant to ibrutinib.

In some embodiments, the renal cell carcinoma is clear cell renal cell carcinoma. In some embodiments, the urothelial carcinoma is a transitional cell urothelial carcinoma. In some embodiments, the colorectal cancer or carcinoma is a K-RAS wild-type EGFR-expressing colorectal cancer or carcinoma.

### BTK Inhibitor Compounds

The BTK inhibitor compound described herein (i.e., ibrutinib) is selective for BTK and kinases having a cysteine residue in an amino acid sequence position of the tyrosine kinase that is homologous to the amino acid sequence position of cysteine 481 in BTK. The BTK inhibitor compound can form a covalent bond with Cys 481 of BTK (e.g., via a Michael reaction). BTK inhibitor compounds include ibrutinib, and pharmaceutically acceptable salts and solvates thereof.

In some embodiments, the BTK inhibitor is a compound of Formula (A) having the structure:

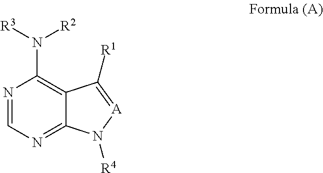

wherein
A is independently selected from N or CR⁵;
R¹ is H, L²-(substituted or unsubstituted alkyl), L²-(substituted or unsubstituted cycloalkyl), L²-(substituted or unsubstituted alkenyl), L²-(substituted or unsubstituted cycloalkenyl), L²-(substituted or unsubstituted heterocycle), L²-(substituted or unsubstituted heteroaryl), or L²-(substituted or unsubstituted aryl), where L² is a bond, 0, S, S(═O), S(═O)₂, C(═O), NHC(═O), C(═O)NH, -(substituted or unsubstituted C₁-C₆ alkylene), or -(substituted or unsubstituted C₂-C₆ alkenylene);
R² and R³ are independently selected from H, lower alkyl and substituted lower alkyl;
R⁴ is L³-X-L⁴-G, wherein,
   L³ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
   X is optional, and when present is a bond, O, C(═O), S, S(═O), S(═O)₂, NH, NR⁹, NHC(O), C(O)NH, NR⁹C(O), C(O)NR⁹, S(═O)₂NH, NHS(═O)₂, S(═O)₂NR⁹, NR⁹S(═O)₂, OC(O)NH, NHC(O)O, OC(O)NR⁹, NR⁹C(O)O, CH═NO, ON═CH, NR¹⁰C(O)NR¹⁰, heteroaryl, aryl, NR¹⁰C(═NR¹¹)NR¹⁰, NR¹⁰C(═NR¹¹), C(═NR¹¹)NR¹⁰, OC(═NR¹¹), or C(═NR¹¹)O;
   L⁴ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
   or L³, X and L⁴ taken together form a nitrogen containing heterocyclic ring;
   G is

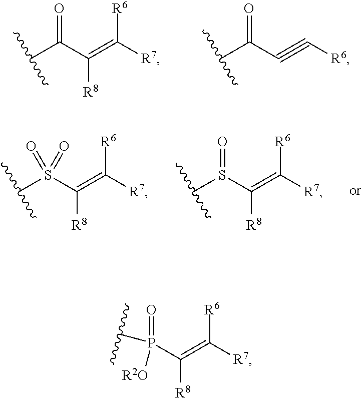

wherein,
R⁶, R⁷ and R⁸ are independently selected from H, lower alkyl or substituted lower alkyl, lower heteroalkyl or substituted lower heteroalkyl, substituted or unsubstituted lower cycloalkyl, and substituted or unsubstituted lower heterocycloalkyl;
R⁵ is H, halogen, —CN, —OH, —NH₂, —SH, substituted or unsubstituted C₁-C₆alkyl, substituted or unsubstituted C₁-C₄alkoxy, substituted or unsubstituted C₁-C₆heteroalkyl, substituted or unsubstituted phenyl, substituted or unsubstituted heteroaryl or substituted or unsubstituted C₃-C₈cycloalkyl;
each R⁹ is independently selected from H, substituted or unsubstituted lower alkyl, and substituted or unsubstituted lower cycloalkyl;
each R¹⁰ is independently H, substituted or unsubstituted lower alkyl, or substituted or unsubstituted lower cycloalkyl; or
two R¹⁰ groups can together form a 5-, 6-, 7-, or 8-membered heterocyclic ring; or
R⁹ and R¹⁰ can together form a 5-, 6-, 7-, or 8-membered heterocyclic ring; or
each R¹¹ is independently selected from H, —S(═O)₂R⁸, —S(═O)₂NH₂, —C(O)R⁸, —CN, —NO₂, heteroaryl, or heteroalkyl; or
   a pharmaceutically acceptable solvate, or pharmaceutically acceptable salt thereof.

In some embodiments, the BTK inhibitor is a compound of Formula (B) having the structure:

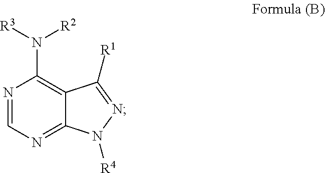

wherein:

R¹ is phenyl-O-phenyl or phenyl-S-phenyl;

R² and R³ are independently H;

R⁴ is L³-X-L⁴-G, wherein,

L³ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR⁹—, —NHC(O)—, —C(O)NH—, —NR⁹C(O)—, —C(O)NR⁹—, —S(═O)₂NH—, —NHS(═O)₂—, —S(═O)₂NR⁹—, —NR⁹S(═O)₂—, —OC(O)NH—, —NHC(O)O—, —OC(O)NR⁹—, —NR⁹C(O)O—, —CH═NO—, —ON═CH—, —NR¹⁰C(O)NR¹⁰—, heteroaryl-, aryl-, —NR¹⁰C(═NR¹¹)NR¹⁰—, —NR¹⁰C(═NR¹¹)—, —C(═NR¹¹)NR¹⁰—, —OC(═NR¹¹)—, or —C(═NR¹¹)O—;

L⁴ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L³, X and L⁴ taken together form a nitrogen containing heterocyclic ring;

G is

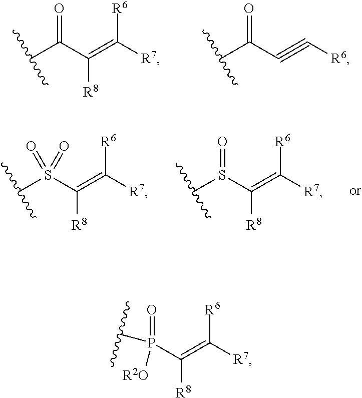

wherein,

R⁶, R⁷ and R⁸ are independently selected from H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, and substituted or unsubstituted heteroaryl;

each R⁹ is independently selected from H, substituted or unsubstituted lower alkyl, and substituted or unsubstituted lower cycloalkyl;

each R¹⁰ is independently H, substituted or unsubstituted lower alkyl, or substituted or unsubstituted lower cycloalkyl; or

two R¹⁰ groups can together form a 5-, 6-, 7-, or 8-membered heterocyclic ring; or R¹⁰ and R¹¹ can together form a 5-, 6-, 7-, or 8-membered heterocyclic ring; or each R¹¹ is independently selected from H or substituted or unsubstituted alkyl; or a pharmaceutically acceptable salt thereof. In some embodiments, L³, X and L⁴ taken together form a nitrogen containing heterocyclic ring. In some embodiments, the nitrogen containing heterocyclic ring is a piperidine group. In some embodiments, G is

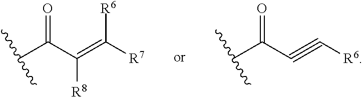

In some embodiments, the compound of Formula (A) or (B) is 1-[(3R)-3-[4-amino-3-(4-phenoxyphenyl)pyrazolo[3,4-d]pyrimidin-1-yl]piperidin-1-yl]prop-2-en-1-one.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or ibrutinib or any other suitable name refers to the compound with the following structure:

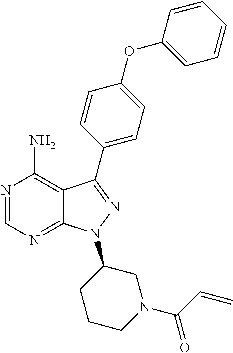 [SMILES: C=CC(=O)N1CCC[C@@H](n2nc(-c3ccc(Oc4ccccc4)cc3)c3c(N)ncnc32)C1]

A wide variety of pharmaceutically acceptable salts may be formed from ibrutinib and includes:
acid addition salts formed by reacting ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

In some embodiments, the BTK inhibitor is a compound of Formula (C) having the structure:

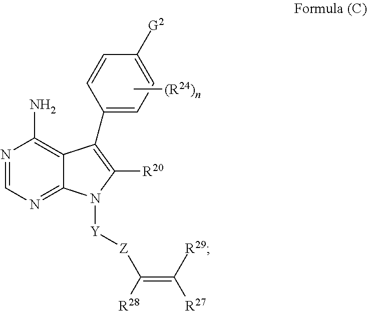

wherein:
R²⁰ is halogen, —CN, —OH, —NH₂, —SH, substituted or unsubstituted C₁-C₆alkyl, substituted or unsubstituted C₁-C₄alkoxy, substituted or unsubstituted C₁-C₆heteroalkyl, substituted or unsubstituted phenyl, substituted or unsubstituted heteroaryl or substituted or unsubstituted C₃-C₈cycloalkyl;
G² is substituted or unsubstituted C₂-C₄alkenyl, substituted or unsubstituted C₂-C₄alkynyl, substituted or unsubstituted C₃-C₈cycloalkyl, substituted or unsubstituted C₁-C₄alkoxy, substituted or unsubstituted C₁-C₄heteroalkyl, substituted or unsubstituted C₂-C₇heterocycloalkyl, halogen, —CN, —NO₂, —OH, —OCF₃, —OCH₂F, —OCF₂H, —CF₃, —SCH₃, —N(R²¹)S(═O)₂R²³, —S(═O)₂N(R²¹)(R²²), —S(═O)R²³, —S(═O)₂R²³, —C(═O)R²³, —OC(═O)R²³, —CO₂R²¹, —N(R²¹)(R²²), —C(═O)N(R²¹)(R²²), —N(R²¹)C(═O)R²³, —N(R²¹)C(═O)OR²², —N(R²¹)C(═O)N(R²¹)(R²²), or L^{a}-A²;
L^{a} is a bond, —CH₂—, —CH(OH)—, —C(O)—, —CH₂O—, —OCH₂—, —SCH₂, —CH₂S—, —N(R²¹)—, —N(R²¹)C(O)—, —C(O)N(R²¹)—, —N(R²¹)C(O)N(R²¹)—, —O—, —S—, —S(O)—, —S(O)₂—, —N(R²¹)S(O)₂—, or —S(O)₂N(R²¹)—;
A² is a substituted or unsubstituted cycloalkyl, substituted or unsubstituted heterocycloalkyl, substituted or unsubstituted aryl, or a substituted or unsubstituted heteroaryl;
each R²⁴ is each independently halogen, —CN, —NO₂, —OH, —OCF₃, —OCH₂F, —OCF₂H, —CF₃, —SCH₃, —N(R²¹)S(═O)₂R²³, —S(═O)₂N(R²¹)(R²²), —S(═O)R²³, —S(═O)₂R²³, —C(═O)R²³, —OC(═O)R²³, —CO₂R²¹, —N(R²¹)(R²²), —C(═O)N(R²¹)(R²²), —N(R²¹)C(═O)R²³, —N(R²¹)C(═O)OR²², —N(R²¹)C(═O)N(R²¹)(R²²), substituted or unsubstituted alkyl, substituted or unsubstituted alkoxy, substituted or unsubstituted heteroalkyl, substituted or unsubstituted heterocycloalkyl, or substituted or unsubstituted cycloalkyl; each R²¹ and R²² are each independently H, substituted or unsubstituted C₁-C₆alkyl, or substituted or unsubstituted C₃-C₈cycloalkyl;
each R²³ is each independently substituted or unsubstituted C₁-C₆alkyl, or substituted or unsubstituted C₃-C₈cycloalkyl;
n is 0-4;
Y is an optionally substituted group selected from C₁-C₆alkylene, C₁-C₆heteroalkylene, C₆-C₁₂arylene, C₃-C₁₂heteroarylene, C₁-C₆alkyleneC₆-C₁₂arylene, C₁-C₆alkyleneC₃-C₁₂heteroarylene, C₁-C₆alkyleneC₃-C₈cycloalkylene, C₁-C₆alkyleneC₂-C₇heterocycloalkylene, C₃-C₈cycloalkylene, C₂-C₇heterocycloalkylene, fused C₃-C₈cycloalkyleneC₂-C₇heterocycloalkylene, and spiro C₃-C₈cycloalkyleneC₂-C₇heterocycloalkylene;
Z is —C(═O), —N(R^{a})C(═O), —S(═O)ₓ, or —N(R^{a})S(═O)ₓ, where x is 1 or 2, and R^{a} is H, substituted or unsubstituted C₁-C₆alkyl, or substituted or unsubstituted C₃-C₈cycloalkyl; R²⁷ and R²⁸ are independently H or L-J-W; or R²⁷ and R²⁸ taken together form a bond; L and J are each independently a bond, substituted or unsubstituted C₁-C₆alkylene, substituted or unsubstituted C₃-C₈cycloalkylene, substituted or unsubstituted C₁-C₆heteroalkylene, substituted or unsubstituted C₂-C₇heterocycloalkylene, substituted or unsubstituted C₆-C₁₂arylene, substituted or unsubstituted C₃-C₁₂heteroarylene, —CO—, —O—, or —S—;
R²⁹ is H or L-J-W;
W is H, or NR²⁵R²⁶; and
R²⁵ and R²⁶ are each independently H, substituted or unsubstituted C₁-C₆alkyl, substituted or unsubstituted C₃-C₈cycloalkyl, substituted or unsubstituted C₁-C₆heteroalkyl, substituted or unsubstituted C₂-C₇heterocycloalkyl, substituted or unsubstituted C₆-C₁₂aryl, or substituted or unsubstituted C₃-C₁₂heteroaryl; or
a pharmaceutically acceptable solvate or pharmaceutically acceptable salt thereof.

In some embodiments, G² is L^{a}-A². In some embodiments, L^{a} is —O—; and A² is phenyl. In some embodiments, L^{a} is —OCH₂—. In some embodiments, A² is phenyl.

In some embodiments, Y is optionally substituted C₂-C₇heterocycloalkylene. In some embodiments, Z is —C(═O). In some embodiments, R²⁷, R²⁸, and R²⁹ are H. In some embodiments, R²⁸ and R²⁹ are H; R²⁷ is L-J-W. In some embodiments, L is a bond, substituted or unsubstituted C₁-C₆ alkylene, or substituted or unsubstituted C₃-C₈cycloalkylene; and J is a bond, substituted or unsubstituted C₁-C₆alkylene, substituted or unsubstituted C₃-C₈cycloalkylene, substituted or unsubstituted C₁-C₆heteroalkylene, substituted or unsubstituted C₂-C₇heterocycloalkylene, substituted or unsubstituted C₆-C₁₂arylene, or substituted or unsubstituted C₃-C₁₂ heteroarylene. In some embodiments, L is a bond; J is —CH₂—; and W is NR²⁵R²⁶. In some embodiments, R²⁵ is H, substituted or unsubstituted C₁-C₆alkyl, or substituted or unsubstituted C₃-C₈cycloalkyl; and R²⁶ is substituted or unsubstituted C₁-C₆alkyl, substituted or unsubstituted C₃-C₈cycloalkyl, substituted or unsubstituted C₁-C₆heteroalkyl, substituted or unsubstituted C₂-C₇heterocycloalkyl, substituted or unsubstituted C₆-C₁₂aryl, or substituted or unsubstituted C₃-C₁₂heteroaryl. In some embodiments, R²⁵ and R²⁶ are —CH₃. In some embodiments, R²⁵ and R²⁶ are cyclopropyl. In some embodiments, R²⁵ is —CH₃ and R²⁶ is cyclopropyl.

In some embodiments, Y is optionally substituted C₃-C₈ cycloalkylene. In some embodiments, Z is C(═O), NHC(═O), or N(CH₃)C(═O). In some embodiments, R²⁷, R²⁸, and R²⁹ are H. In some embodiments, R²⁸ and R²⁹ are H; R²⁷ is L-J-W. In some embodiments, L is a bond, substituted or unsubstituted C₁-C₆ alkylene, or substituted or unsubstituted C₃-C₈cycloalkylene; and J is a bond, substituted or unsubstituted C₁-C₆ alkylene, substituted or unsubstituted C₃-C₈cycloalkylene, substituted or unsubstituted C₁-C₆ heteroalkylene, substituted or unsubstituted C₂-C₇heterocycloalkylene, substituted or unsubstituted C₆-C₁₂arylene, or substituted or unsubstituted C₃-C₁₂heteroarylene. In some embodiments, L is a bond; J is —CH₂—; and W is NR²⁵R²⁶. In some embodiments, R²⁵ is H, substituted or unsubstituted C₁-C₆alkyl, or substituted or unsubstituted C₃-C₈cycloalkyl; and R²⁶ is substituted or unsubstituted C₁-C₆alkyl, substituted or unsubstituted C₃-C₈cycloalkyl, substituted or unsubstituted C₁-C₆ heteroalkyl, substituted or unsubstituted C₂-C₇heterocycloalkyl, substituted or unsubstituted C₆-C₁₂aryl, or substituted or unsubstituted C₃-C₁₂heteroaryl. In some embodiments, R²⁵ and R²⁶ are —CH₃. In some embodiments, R²⁵ and R²⁶ are cyclopropyl. In some embodiments, R²⁵ is —CH₃ and R²⁶ is cyclopropyl.

In some embodiments, Y is optionally substituted C₆-C₁₂ arylene. In some embodiments, Y is phenyl. In some embodiments, wherein Z is C(═O), NHC(═O), or N(CH₃)C(═O). In some embodiments, R²⁷, R²⁸, and R²⁹ are H. In some embodiments, R²⁸ and R²⁹ are H; R²⁷ is L-J-W. In some embodiments, L is a bond, substituted or unsubstituted C₁-C₆alkylene, or substituted or unsubstituted C₃-C₈cycloalkylene; and J is a bond, substituted or unsubstituted C₁-C₆alkylene, substituted or unsubstituted C₃-C₈cycloalkylene, substituted or unsubstituted C₁-C₆heteroalkylene, substituted or unsubstituted C₂-C₇heterocycloalkylene, substituted or unsubstituted C₆-C₁₂arylene, or substituted or unsubstituted C₃-C₁₂heteroarylene. In some embodiments, L is a bond; J is —CH₂—; and W is NR²⁵R²⁶. In some embodiments, R²⁵ is H, substituted or unsubstituted C₁-C₆ alkyl, or substituted or unsubstituted C₃-C₈cycloalkyl; and R²⁶ is substituted or unsubstituted C₁-C₆ alkyl, substituted or unsubstituted C₃-C₈cycloalkyl, substituted or unsubstituted C₁-C₆heteroalkyl, substituted or unsubstituted C₂-C₇heterocycloalkyl, substituted or unsubstituted C₆-C₁₂aryl, or substituted or unsubstituted C₃-C₂heteroaryl. In some embodiments, R²⁵ and R²⁶ are —CH₃. In some embodiments, R²⁵ and R²⁶ are cyclopropyl. In some embodiments, R²⁵ is —CH₃ and R²⁶ is cyclopropyl.

In some embodiments, n is 0. In some embodiments, R²⁰ is —F, —Cl, —CH₃, or —OCH₃.

In some embodiments, the BTK inhibitor is a compound of Formula (D) having the structure:

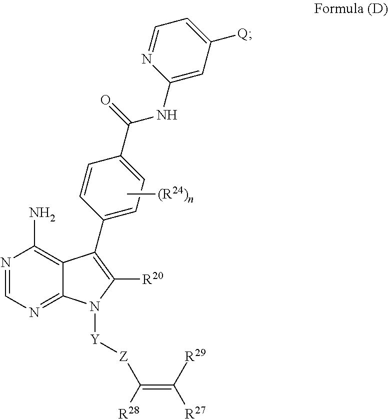

wherein:
R²⁰ is halogen, —CN, —OH, —NH₂, —SH, —CO₂H, substituted or unsubstituted C₁-C₆alkyl, substituted or unsubstituted C₂-C₄alkynyl, substituted or unsubstituted C₁-C₄alkoxy, substituted or unsubstituted C₁-C₆heteroalkyl, substituted or unsubstituted phenyl, substituted or unsubstituted heteroaryl, substituted or unsubstituted C₃-C₈cycloalkyl, or —C(═O)N((R²¹)(R²²);
Q is substituted or unsubstituted C₁-C₆alkyl, C₁-C₆haloalkyl, or —CN;
each R²¹ and R²² are independently H, substituted or unsubstituted C₁-C₆alkyl, or substituted or unsubstituted C₃-C₈cycloalkyl;
each R²³ is independently substituted or unsubstituted C₁-C₆alkyl, or substituted or unsubstituted C₃-C₈cycloalkyl;
each R²⁴ is independently halogen, —CN, —NO₂, —OH, —OCF₃, —OCH₂F, —OCF₂H, —CF₃, —SCH₃, —N(R²¹)S(═O)₂R²³, —S(═O)₂N(R²¹)(R²²), —S(═O)R²³, —S(═O)₂R²³, —C(═O)R²³, —OC(═O)R²³, —CO₂R²¹, —N(R²¹)(R²²), —C(═O)N(R²¹)(R²²), —N(R²¹)C(═O)R²³, —N(R²¹)C(═O)OR²², —N(R²¹)C(═O)N(R²¹)(R²²), substituted or unsubstituted alkyl, substituted or unsubstituted alkoxy, substituted or unsubstituted heteroalkyl, substituted or unsubstituted heterocycloalkyl, or substituted or unsubstituted cycloalkyl;
n is 0-4;
Y is an optionally substituted group selected from C₁-C₆alkylene, C₁-C₆heteroalkylene, C₆-C₁₂arylene, C₃-C₁₂heteroarylene, C₁-C₆alkyleneC₆-C₁₂arylene, C₁-C₆alkyleneC₃-C₁₂heteroarylene, C₁-C₆alkyleneC₃-C₈cycloalkylene, C₁-C₆alkyleneC₂-C₇heterocycloalkylene, C₃-C₈cycloalkylene, C₂-C₇heterocycloalkylene, fused C₃-C₈cycloalkyleneC₂-C₇heterocycloalkylene, and spiro C₃-C₈cycloalkyleneC₂-C₇heterocycloalkylene;
Z is —C(═O), —N(R^{a})C(═O), —S(═O)ₓ, or —N(R^{a})S(═O)ₓ, where x is 1 or 2, and R^{a} is H, substituted or unsubstituted C₁-C₆alkyl, or substituted or unsubstituted C₃-C₈cycloalkyl;
R²⁷ and R²⁸ are independently H or L-J-W; or R²⁷ and R²⁸ taken together form a bond; L and J are each independently a bond, substituted or unsubstituted C₁-C₆alkylene, substituted or unsubstituted C₃-C₈cycloalkylene, substituted or unsubstituted C₁-C₆heteroalkylene, substituted or unsubstituted C₂-C₇heterocycloalkylene, substituted or unsubstituted C₆-C₁₂arylene, substituted or unsubstituted C₃-C₁₂heteroarylene, —CO—, —O—, or —S—;
R²⁹ is H or L-J-W;
W is H, or NR²⁵R²⁶; and
R²⁵ and R²⁶ are each independently H, substituted or unsubstituted C₁-C₆alkyl, substituted or unsubstituted C₃-C₈cycloalkyl, substituted or unsubstituted C₁-C₆heteroalkyl, substituted or unsubstituted C₂-C₇heterocycloalkyl, substituted or unsubstituted C₆-C₁₂aryl, or substituted or unsubstituted C₃-C₁₂heteroaryl; or
a pharmaceutically acceptable solvate or pharmaceutically acceptable salt thereof.

In some embodiments, the BTK inhibitor is selected from the group consisting of:

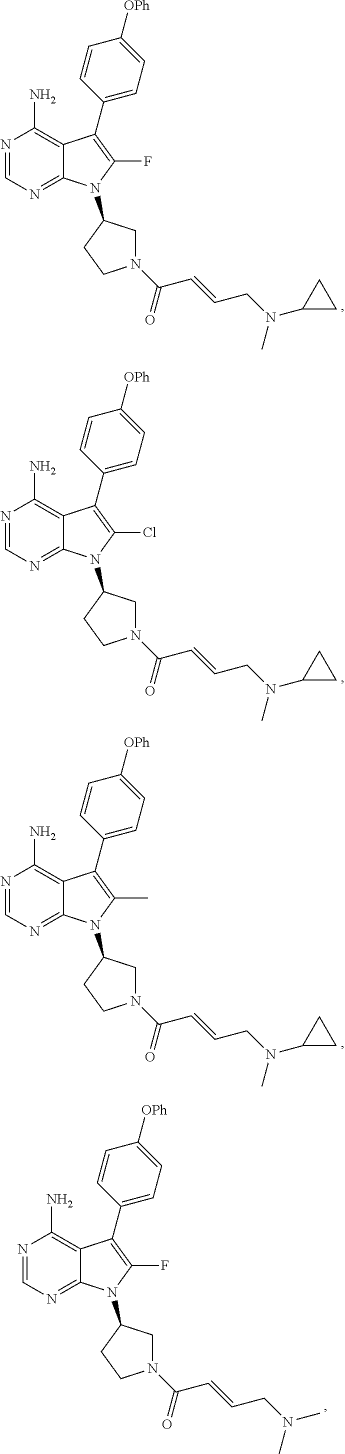 [SMILES: CN(C/C=C/C(=O)N1CC[C@@H](n2c(F)c(-c3ccc(Oc4ccccc4)cc3)c3c(N)ncnc32)C1)C1CC1 ; CN(C/C=C/C(=O)N1CC[C@@H](n2c(Cl)c(-c3ccc(Oc4ccccc4)cc3)c3c(N)ncnc32)C1)C1CC1 ; Cc1c(-c2ccc(Oc3ccccc3)cc2)c2c(N)ncnc2n1[C@@H]1CCN(C(=O)/C=C/CN(C)C2CC2)C1 ; CN(C)C/C=C/C(=O)N1CC[C@@H](n2c(F)c(-c3ccc(Oc4ccccc4)cc3)c3c(N)ncnc32)C1]

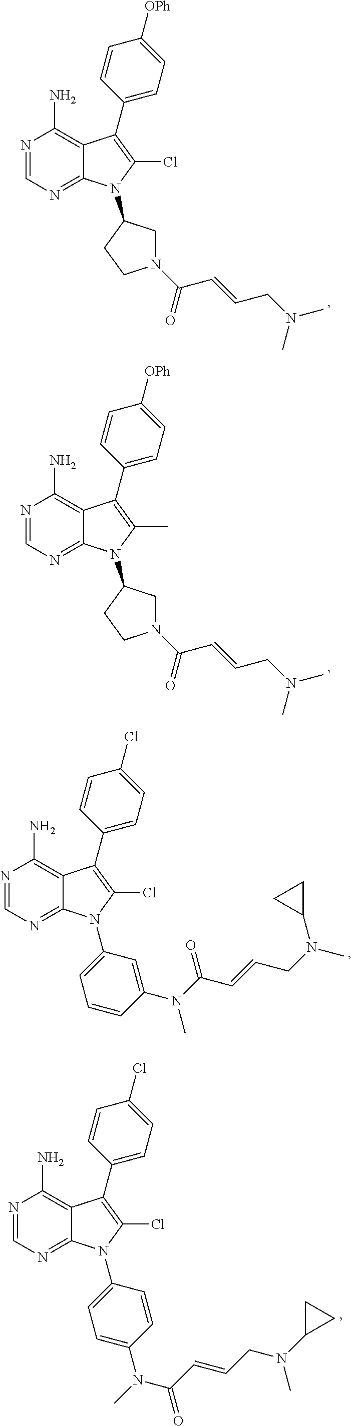 [SMILES: CN(C)C/C=C/C(=O)N1CC[C@@H](n2c(Cl)c(-c3ccc(Oc4ccccc4)cc3)c3c(N)ncnc32)C1 ; Cc1c(-c2ccc(Oc3ccccc3)cc2)c2c(N)ncnc2n1[C@@H]1CCN(C(=O)/C=C/CN(C)C)C1 ; CN(C(=O)/C=C/CN(C)C1CC1)c1cccc(-n2c(Cl)c(-c3ccc(Cl)cc3)c3c(N)ncnc32)c1 ; CN(C(=O)/C=C/CN(C)C1CC1)c1ccc(-n2c(Cl)c(-c3ccc(Cl)cc3)c3c(N)ncnc32)cc1]

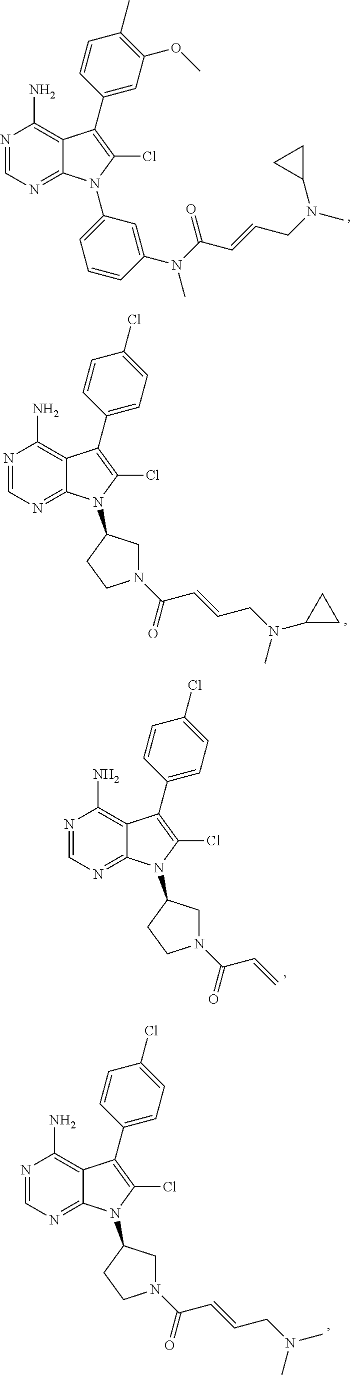

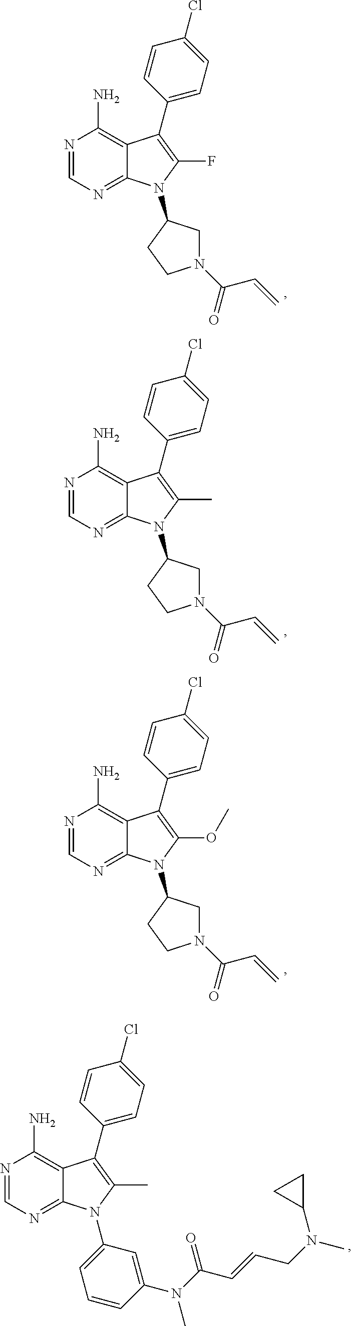

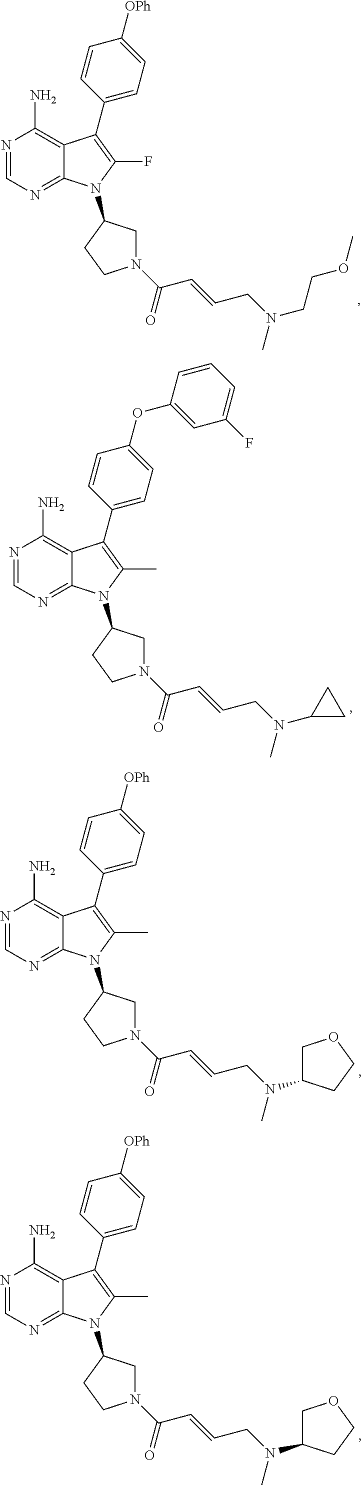

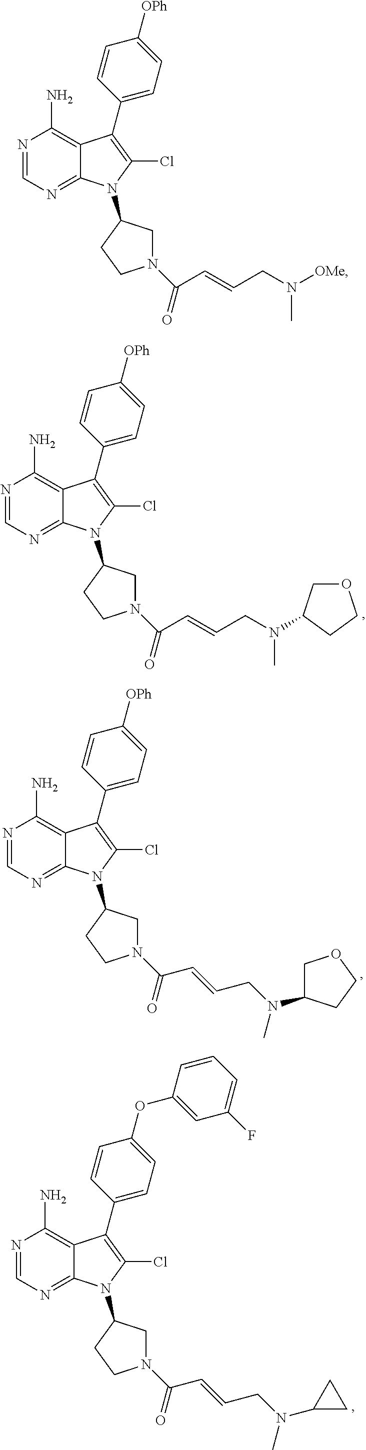

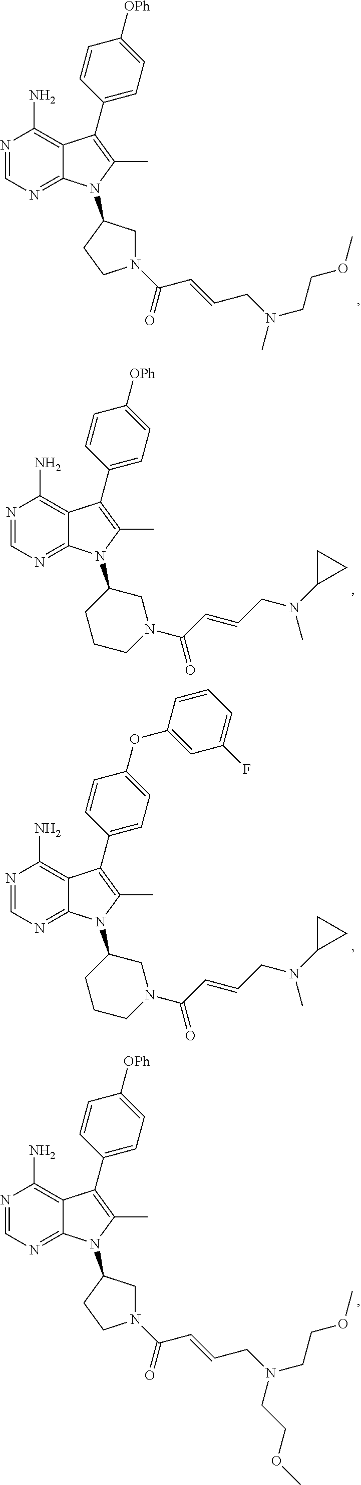

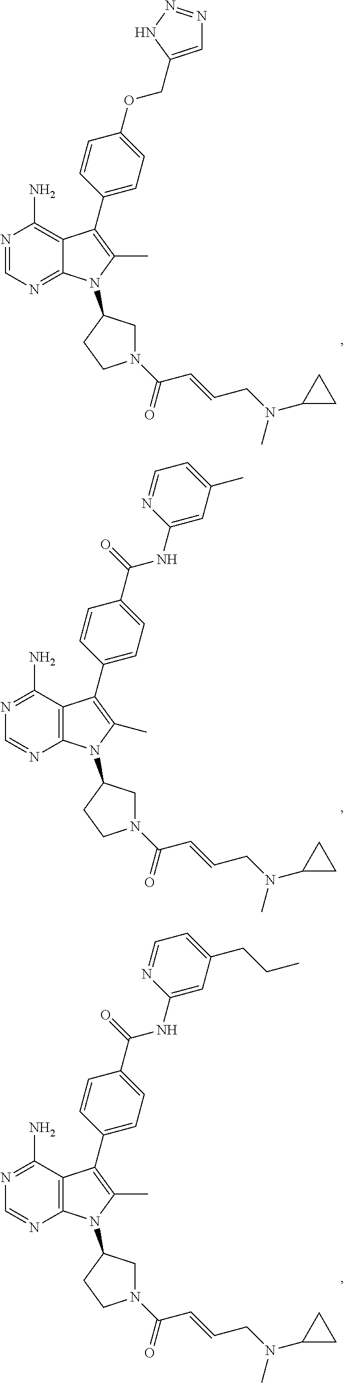

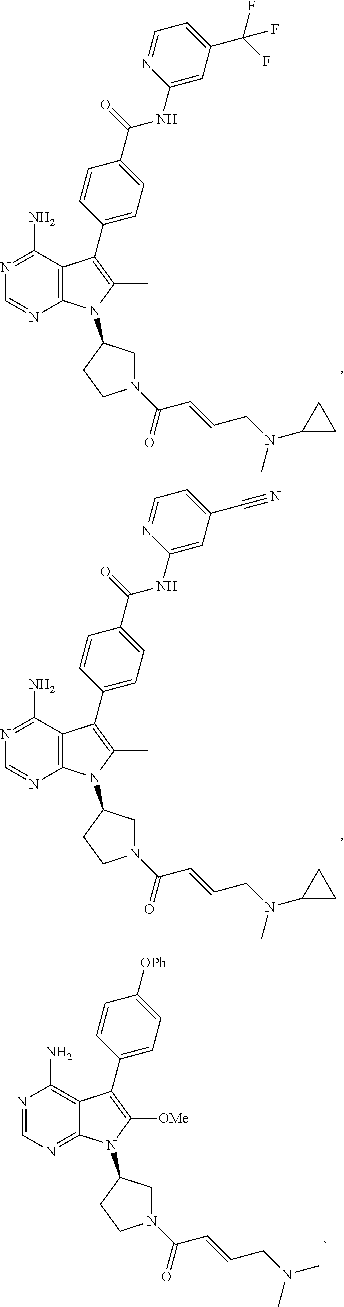

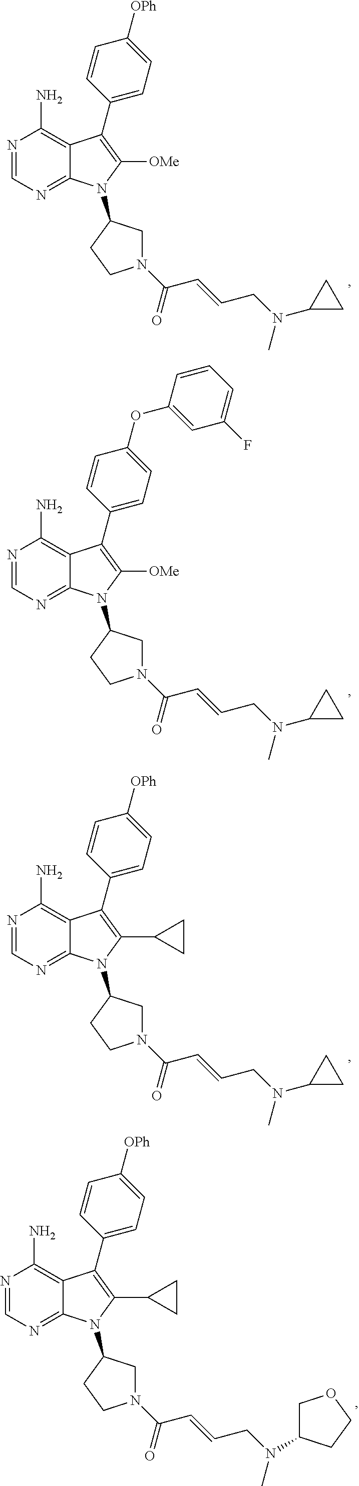 [SMILES: COc1c(-c2ccc(Oc3ccccc3)cc2)c2c(N)ncnc2n1[C@@H]1CCN(C(=O)/C=C/CN(C)C2CC2)C1 ; COc1c(-c2ccc(Oc3cccc(F)c3)cc2)c2c(N)ncnc2n1[C@@H]1CCN(C(=O)/C=C/CN(C)C2CC2)C1 ; CN(C/C=C/C(=O)N1CC[C@@H](n2c(C3CC3)c(-c3ccc(Oc4ccccc4)cc3)c3c(N)ncnc32)C1)C1CC1 ; CN(C/C=C/C(=O)N1CC[C@@H](n2c(C3CC3)c(-c3ccc(Oc4ccccc4)cc3)c3c(N)ncnc32)C1)[C@H]1CCOC1]

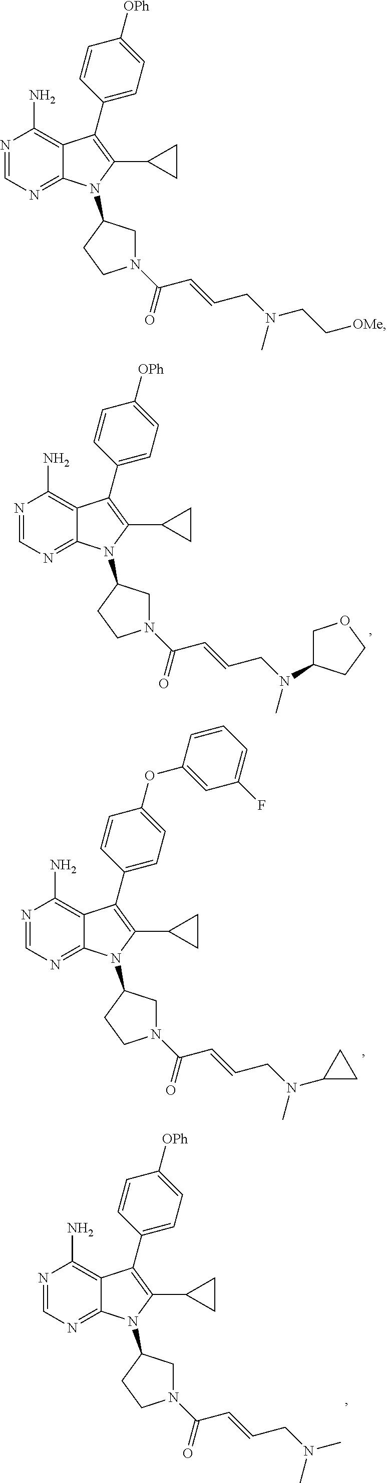

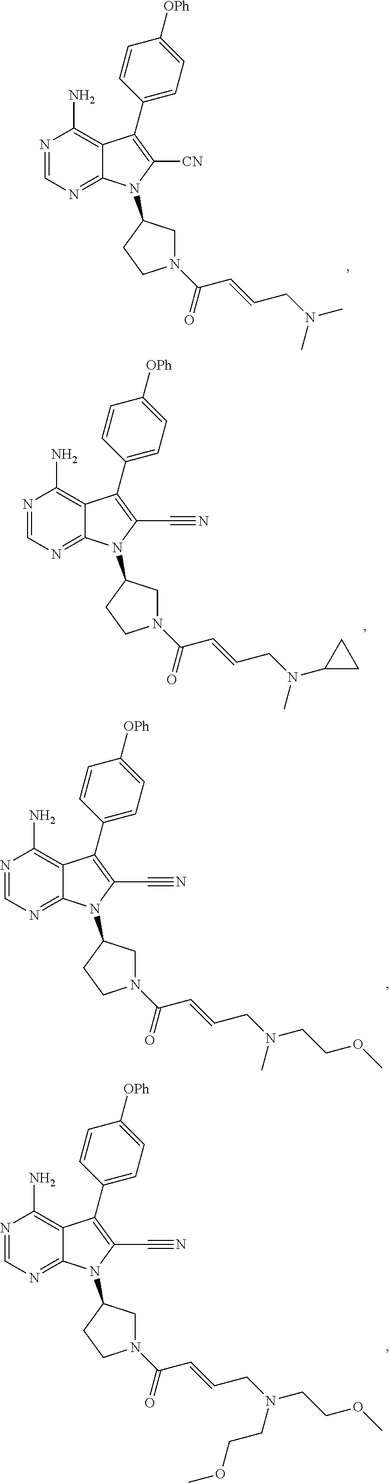 [SMILES: CN(C)C/C=C/C(=O)N1CC[C@@H](n2c(C#N)c(-c3ccc(Oc4ccccc4)cc3)c3c(N)ncnc32)C1 ; CN(C/C=C/C(=O)N1CC[C@@H](n2c(C#N)c(-c3ccc(Oc4ccccc4)cc3)c3c(N)ncnc32)C1)C1CC1 ; COCCN(C)C/C=C/C(=O)N1CC[C@@H](n2c(C#N)c(-c3ccc(Oc4ccccc4)cc3)c3c(N)ncnc32)C1 ; COCCN(C/C=C/C(=O)N1CC[C@@H](n2c(C#N)c(-c3ccc(Oc4ccccc4)cc3)c3c(N)ncnc32)C1)CCOC]

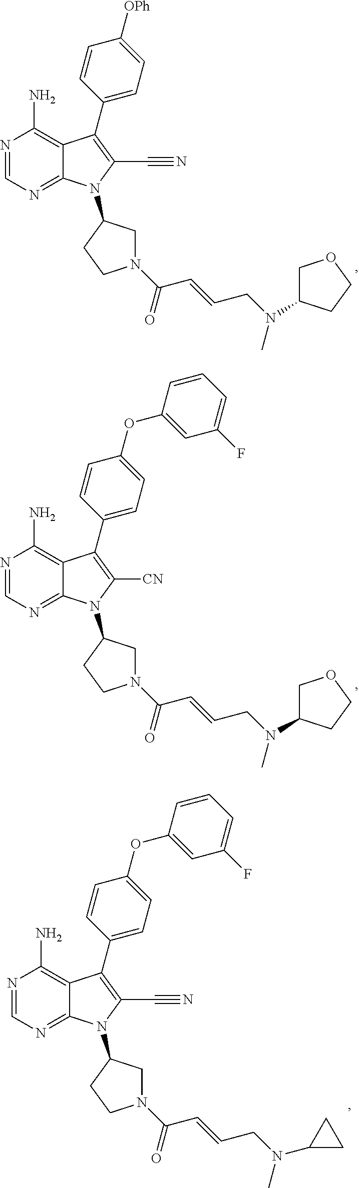 [SMILES: CN(C/C=C/C(=O)N1CC[C@@H](n2c(C#N)c(-c3ccc(Oc4ccccc4)cc3)c3c(N)ncnc32)C1)[C@H]1CCOC1 ; CN(C/C=C/C(=O)N1CC[C@@H](n2c(C#N)c(-c3ccc(Oc4cccc(F)c4)cc3)c3c(N)ncnc32)C1)[C@@H]1CCOC1 ; CN(C/C=C/C(=O)N1CC[C@@H](n2c(C#N)c(-c3ccc(Oc4cccc(F)c4)cc3)c3c(N)ncnc32)C1)C1CC1]

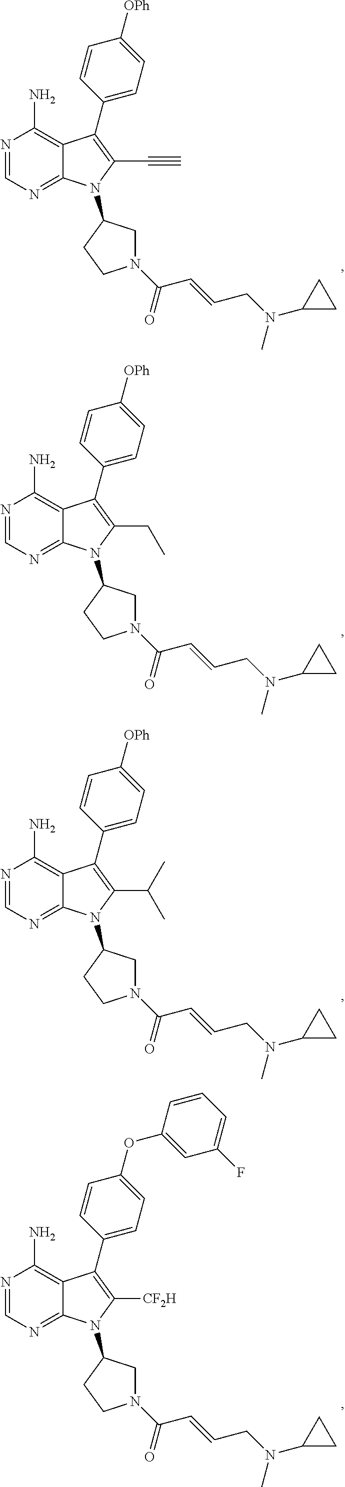 [SMILES: C#Cc1c(-c2ccc(Oc3ccccc3)cc2)c2c(N)ncnc2n1[C@@H]1CCN(C(=O)/C=C/CN(C)C2CC2)C1 ; CCc1c(-c2ccc(Oc3ccccc3)cc2)c2c(N)ncnc2n1[C@@H]1CCN(C(=O)/C=C/CN(C)C2CC2)C1 ; CC(C)c1c(-c2ccc(Oc3ccccc3)cc2)c2c(N)ncnc2n1[C@@H]1CCN(C(=O)/C=C/CN(C)C2CC2)C1 ; CN(C/C=C/C(=O)N1CC[C@@H](n2c(C(F)F)c(-c3ccc(Oc4cccc(F)c4)cc3)c3c(N)ncnc32)C1)C1CC1]

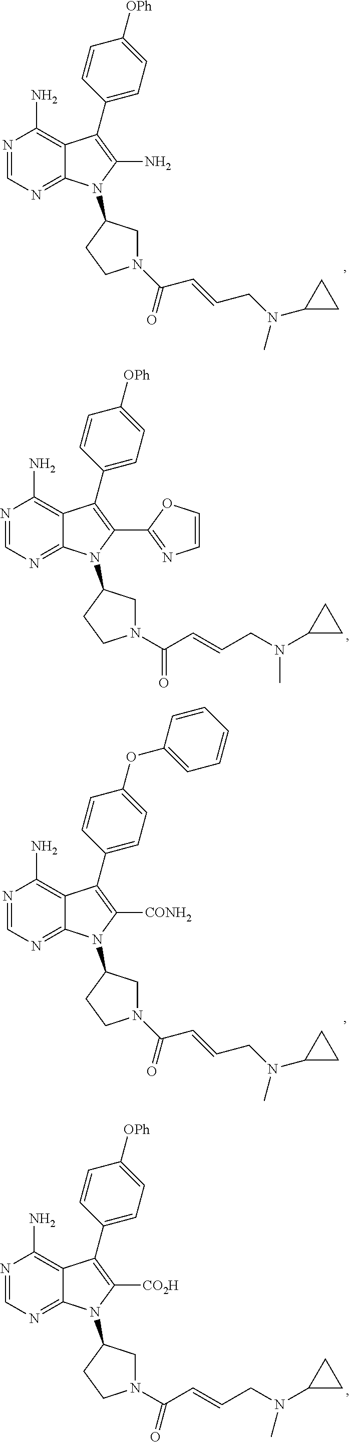

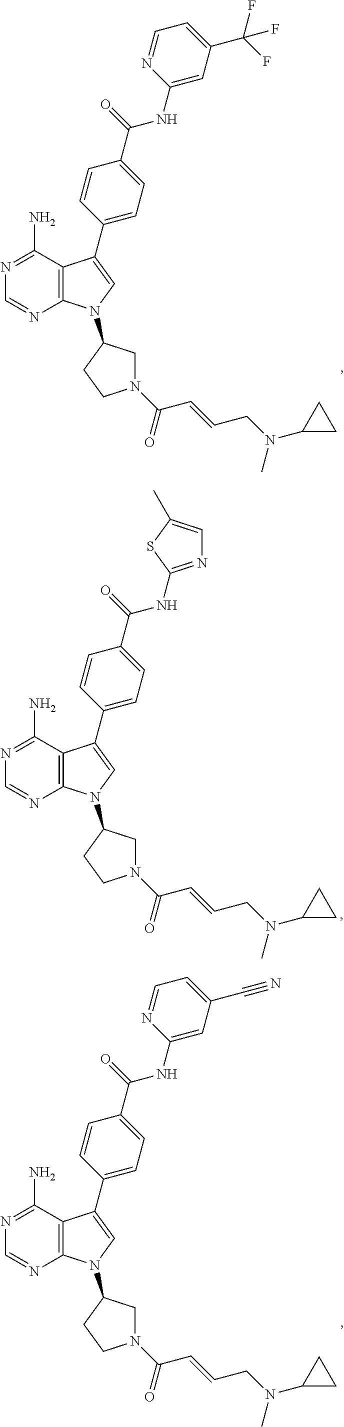

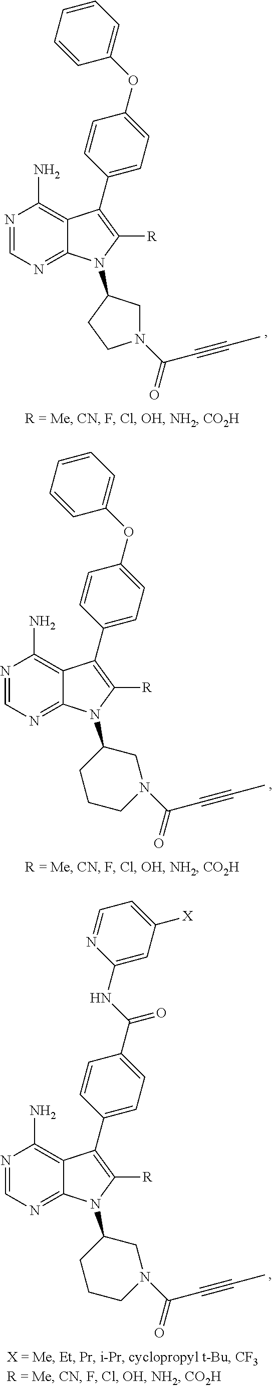

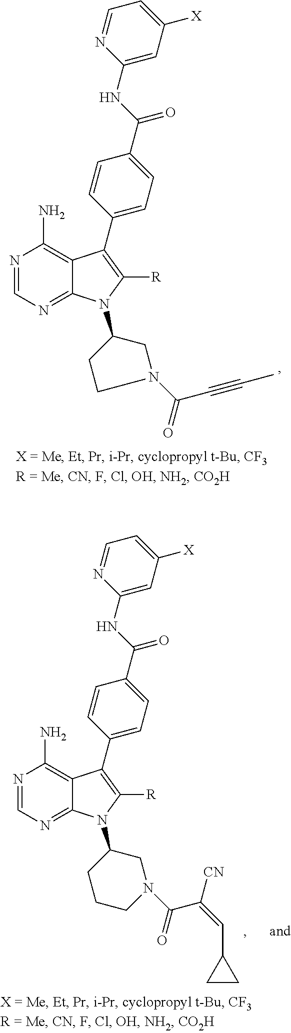

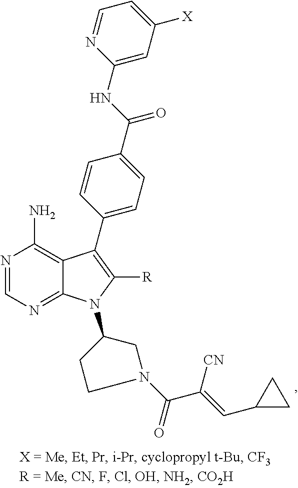

or a pharmaceutically acceptable solvate or pharmaceutically acceptable salt thereof.

The term “pharmaceutically acceptable salts” refers to a salt of a compound, which does not cause significant irritation to a mammal to which it is administered and does not substantially abrogate the biological activity and properties of the compound. Pharmaceutically acceptable salts include acid addition salts formed by a compound and an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like; acid addition salts formed by compound and an inorganic acid, which includes hydrochloric acid, hydrobromic acid, sulfuric acid, nitric acid, phosphoric acid, hydroiodic acid, hydrofluoric acid, phosphorous acid, and the like. Further salts include those in which the counterion is a cation, such as sodium, lithium, potassium, calcium, magnesium, ammonium, and quaternary ammonium (substituted with at least one organic moiety) cations.

Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of a compound, or pharmaceutically acceptable salts thereof, are conveniently prepared or formed during the processes described herein or methods known in the art. In some embodiments, solvates of a compound are anhydrous. In some embodiments, a compound, or pharmaceutically acceptable salts thereof, exist in unsolvated form. In some embodiments, a compound, or pharmaceutically acceptable salts thereof, exist in unsolvated form and are anhydrous. It should be understood that a reference to a pharmaceutically acceptable salt includes the solvent addition forms (solvates).

In yet other embodiments, ibrutinib, or a pharmaceutically acceptable salt thereof, is prepared in various forms, including but not limited to, amorphous phase, crystalline forms, milled forms and nano-particulate forms. In some embodiments, ibrutinib, or a pharmaceutically acceptable salt thereof, is amorphous. In some embodiments, ibrutinib, or a pharmaceutically acceptable salt thereof, is amorphous and anhydrous. In some embodiments, ibrutinib, or a pharmaceutically acceptable salt thereof, is crystalline. In some embodiments, ibrutinib, or a pharmaceutically acceptable salt thereof, is crystalline and anhydrous.

In some embodiments, ibrutinib is prepared as outlined in U.S. Pat. No. 7,514,444 (incorporated by reference).

In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) or JTE-051 (Japan Tobacco Inc).

In some embodiments, the BTK inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the BTK inhibitor is:

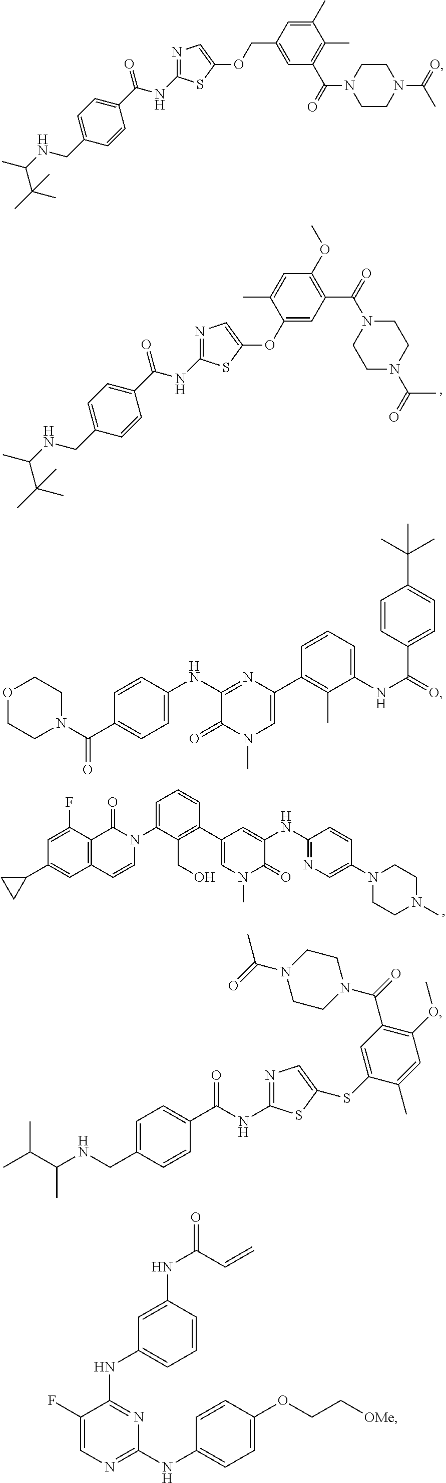

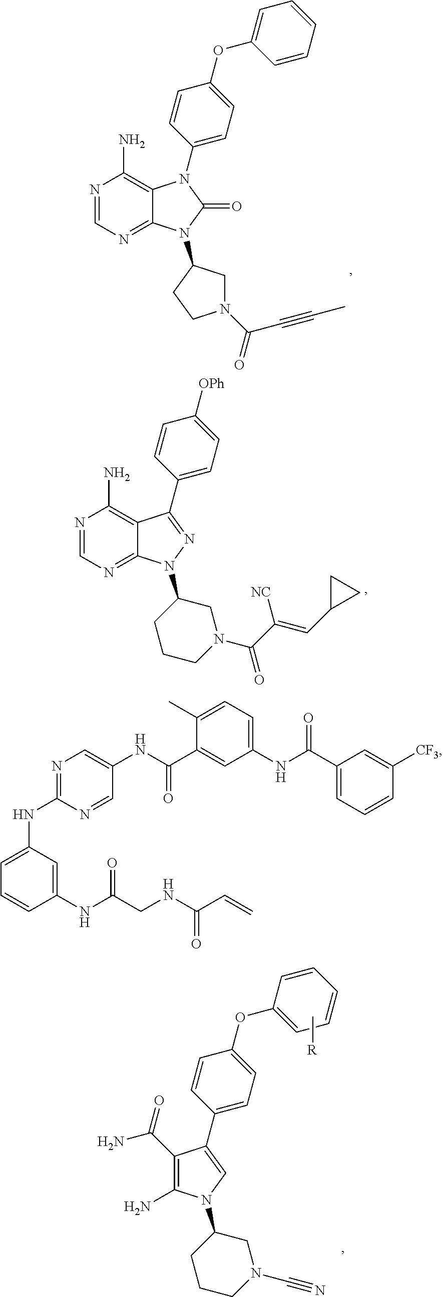

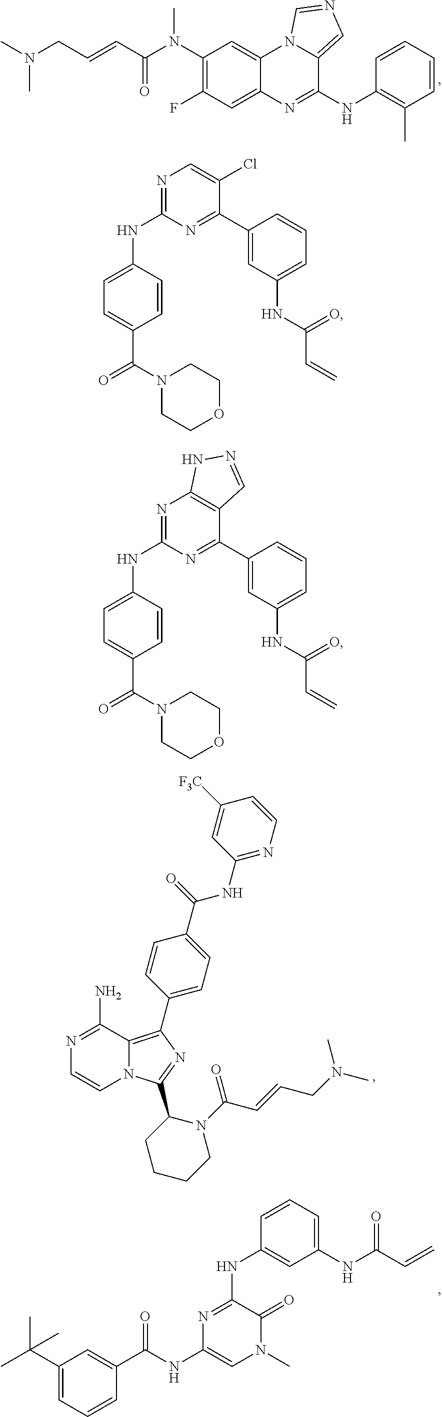

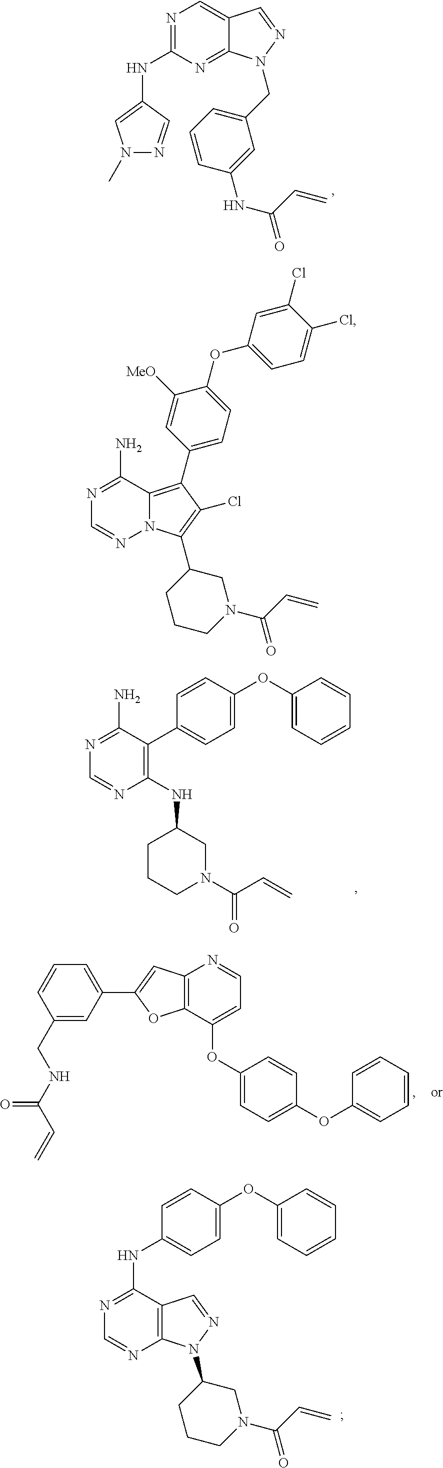

or a pharmaceutically acceptable salt thereof.
mTOR Inhibitors

mTOR inhibitors are inhibitors of the a serine/threonine kinase mammalian Target Of Rapamycin (mTOR). Examples of mTOR inhibitors include deforolimus, everolimus, ridaforolimus, temsirolimus, and sirolimus.

In some embodiments, the mTOR inhibitor that is combined with a BTK inhibitor such as ibrutinib is everolimus or a pharmaceutically acceptable salt thereof. In some embodiments, everolimus is administered at a dosage of about 1-20 mg/day. In some embodiments, everolimus is administered at a dosage of about 20 mg/day. In some embodiments, everolimus is administered at a dosage of about 10 mg/day. In some embodiments, everolimus is administered at a dosage of about 5 mg/day. In some embodiments, everolimus is administered at a dosage of about 2.5 mg/day. In some embodiments, everolimus is administered at a low dosage of less than 2.5 mg/day. In some embodiments, everolimus is administered at a low dosage of about 1-2 mg/day.

In some embodiments, the mTOR inhibitor that is combined with a BTK inhibitor such as ibrutinib is sirolimus or a pharmaceutically acceptable salt thereof. In some embodiments, sirolimus is administered at a dosage of about 2-5 mg/day. In some embodiments, sirolimus is administered at a low dosage of less than 2 mg/day. In some embodiments, sirolimus is administered at a low dosage of about 1 mg/day. In some embodiments, sirolimus is administered a dosage of about 1-15 mg/day to a subject who weighs at least 40 kg. In some embodiments, sirolimus is administered at a loading dosage of about 6 or 15 mg. In some embodiments, sirolimus is administered at a maintanence dosage of about 2-5 mg/day. In some embodiments, sirolimus is administered at a maintanence dosage of about 1 mg/day. In some embodiments, sirolimus is administered at a loading dosage of about 3 mg/m² to a subject who weighs less than 40 kg. In some embodiments, sirolimus is administered at a maintanence dosage of about 1 mg/m²/day. In some embodiments, sirolimus is administered at a low maintanence dosage of less than 1 mg/m²/day.

### Pazopanib

Pazopanib, 5-[[4-[(2,3-Dimethyl-2H-indazol-6-yl)(methyl)amino]pyrimidin-2-yl]amino]-2-methylbenzenesulfonamide monohydrochloride, is an oral angiogenesis inhibitor targeting the tyrosine kinase activity associated with vascular endothelial growth factor receptor (VEGFR)-1, -2 and -3, platelet-derived growth factor receptor (PDGFR)-α, and PDGFR-β, and stem cell factor receptor (c-KIT).

In some embodiments, pazopanib or a salt of pazopanib (e.g., pazopanib HCl), is administered to an individual in combination with a BTK inhibitor. In some embodiments, pazopanib is administered to an individual in combination with ibrutinib. In some embodiments, pazopanib HCl is administered to an individual in combination with ibrutinib.

In some embodiments, pazopanib or a salt of pazopanib (e.g., pazopanib HCl) is administered to the individual continuously, e.g., without drug holidays. In some embodiments, administration of pazopanib or a salt of pazopanib (e.g., pazopanib HCl), is not halted on the days that ibrutinib is not administered (i.e., during an ibrutinib drug holiday). In some embodiments, administration of pazopanib or a salt of pazopanib (e.g., pazopanib HCl) is halted on the days that ibrutinib is not administered (i.e., during an ibrutinib drug holiday).

In some embodiments, pazopanib or a salt of pazopanib (e.g., pazopanib HCl) is administered by an immediate release dosage form. In some embodiments, pazopanib or a salt of pazopanib (e.g., pazopanib HCl) is administered by a controlled release dosage form.

In some embodiments, pazopanib or a salt of pazopanib (e.g., pazopanib HCl) is administered orally (e.g., by capsules or tablets). In some embodiments, pazopanib or a salt of pazopanib (e.g., pazopanib HCl) is administered by an immediate release oral dosage form (e.g., by capsules or tablets). In some embodiments, pazopanib or a salt of pazopanib (e.g., pazopanib HCl) is administered by a controlled release oral dosage form (e.g., by capsules or tablets).

In some embodiments, pazopanib or a salt of pazopanib (e.g., pazopanib HCl) is administered intravenously.

In some embodiments, pazopanib or a salt of pazopanib (e.g., pazopanib HCl) is administered when the individual is in fast mode. In some embodiments, pazopanib or a salt of pazopanib (e.g., pazopanib HCl) is administered at least about 1 hour before a meal. In some embodiments, pazopanib or a salt of pazopanib (e.g., pazopanib HCl) is administered at least about 2 hours after a meal.

In some embodiments, pazopanib or a salt of pazopanib is administered once per day, twice per day, three times per day, or four times per day. In some embodiments, pazopanib or a salt of pazopanib is administered once per day. In some embodiments, pazopanib or a salt of pazopanib is administered twice per day. In some embodiments, pazopanib or a salt of pazopanib is administered three times per day. In some embodiments, pazopanib or a salt of pazopanib is administered four times per day.

In some embodiments, pazopanib or a salt of pazopanib is administered twice per day. In some embodiments, each dose of pazopanib or a salt of pazopanib is administered 4 to 8 hours apart. In some embodiments, any of the methods disclosed herein comprise administering a first dose of pazopanib or a salt of pazopanib and a second dose of pazopanib or a salt of pazopanib, wherein the first dose and the second dose are administered 4 to 8 hours apart.

In some embodiments, pazopanib or a salt of pazopanib is administered three times per day. In some embodiments, each dose of pazopanib or a salt of pazopanib is administered 4 to 8 hours apart. In some embodiments, any of the methods disclosed herein comprise administering a first dose of pazopanib or a salt of pazopanib, a second dose of pazopanib or a salt of pazopanib and a third dose of pazopanib or a salt of pazopanib, wherein the first dose, the second dose and the third dose are administered 4 to 8 hours apart.

In some embodiments, pazopanib or a salt of pazopanib is administered four times per day. In some embodiments, each dose of pazopanib or a salt of pazopanib is administered 4 to 8 hours apart. In some embodiments, any of the methods disclosed herein comprise administering a first dose of pazopanib or a salt of pazopanib, a second dose of pazopanib or a salt of pazopanib, a third dose of pazopanib or a salt of pazopanib, and a fourth dose of pazopanib or a salt of pazopanib, wherein the first dose, the second dose, the third dose and the fourth dose are administered 4 to 8 hours apart.

In some embodiments, the daily dose of pazopanib is about 200 mg to about 800 mg, about 400 mg to about 800 mg, or about 600 mg to about 800 mg. In some embodiments, the daily dose of pazopanib is about 200 mg to about 800 mg. In some embodiments, the daily dose of pazopanib is about 400 mg to about 800 mg. In some embodiments, the daily dose of pazopanib is about 600 mg to about 800 mg.

In some embodiments, the daily dose of pazopanib is about 200 mg, about 400 mg, about 600 mg or about 800 mg. In some embodiments, the daily dose of pazopanib is about 200 mg. In some embodiments, the daily dose of pazopanib is about 400 mg. In some embodiments, the daily dose of pazopanib is about 600 mg. In some embodiments, the daily dose of pazopanib is about 800 mg.

In some embodiments, the daily dose of pazopanib HCl is about 216.7 mg to about 866.8 mg, about 433.4 mg to about 866.8 mg, or about 650.1 mg to about 866.8 mg. In some embodiments, the daily dose of pazopanib HCl is about 216.7 mg to about 866.8 mg. In some embodiments, the daily dose of pazopanib HCl is about 433.4 mg to about 866.8 mg. In some embodiments, the daily dose of pazopanib HCl is about 650.1 mg to about 866.8 mg.

In some embodiments, the daily dose of pazopanib HCl is about 216.7 mg, about 433.4 mg, about 650.1 mg or about 866.8 mg. In some embodiments, the daily dose of pazopanib HCl is about 216.7 mg. In some embodiments, the daily dose of pazopanib HCl is about 433.4 mg. In some embodiments, the daily dose of pazopanib HCl is about 650.1 mg. In some embodiments, the daily dose of pazopanib HCl is about 866.8 mg.

### Paclitaxel and Docetaxel

Paclitaxel and docetaxel are taxanes. Paclitaxel is also known as Taxol®. Docetaxel is also known as Taxotere®. Taxanes are drugs that block cell growth by stopping mitosis. Taxanes interfere with microtubules. A taxane is a type of mitotic inhibitor (or microtubule inhibitor) and a type of antimicrotuble agent. Other exemplary taxanes include cabazitaxel. Protein-bound paclitaxel is known as Abraxane® or nab-paclitaxel. In some embodiments, as used herein, “paclitaxel” may refer to protein-bound paclitaxel as well as paclitaxel. In some embodiments, as used herein, “paclitaxel” does not refer to protein-bound paclitaxel. In some embodiments, paclitaxel may be replaced with nab-paclitaxel in the methods and compositions disclosed herein.

### EGFR Inhibitors

Cetuximab is an epidermal growth factor receptor (EGFR) inhibitor. Cetuximab is also known as Erbitux® or C225. EGFR inhibitors may also be known as HER-1 inhibitors or ErbB-1 inhibitors. EGFR inhibitors are either tyrosine kinase inhibitors or monoclonal antibodies. Additional exemplary EGFR inhibitors include erlotinib (Tarceva®); gefitonib (Iressa®); lapantinib (Tykerb®); panitumumab (Vectibix®), and the like. In some embodiments, cetuximab may be replaced with other EGFR inhibitors in the methods and compositions disclosed herein.

### Additional Combination Therapies

In certain embodiments, (1) a BTK inhibitor and (2) an anticancer agent (e.g., an mTOR inhibitor; pazopanib; paclitaxel; docetaxel; or an EGFR inhibitor) are administered in combination with an additional therapeutic agent for the treatment of a solid tumor. In certain embodiments, a (1) BTK inhibitor (e.g., ibrutinib) and (2) the anticancer agent (e.g., an mTOR inhibitor; pazopanib; paclitaxel; docetaxel; or an EGFR inhibitor) are administered in combination with an additional therapeutic agent for the treatment of a solid tumor. In some embodiments, the additional therapeutic agent comprises an agent selected from: bendamustine, bortezomib, lenalidomide, idelalisib (GS-1101), vorinostat, ofatumumab, everolimus, panobinostat, temsirolimus, romidepsin, vorinostat, fludarabine, cyclophosphamide, mitoxantrone, pentostatine, prednisone, etopside, procarbazine, and thalidomide.

In some embodiments, the additional therapeutic agent is selected from a chemotherapeutic agent, a biologic agent, radiation therapy, bone marrow transplant and surgery. In some embodiments, the chemotherapeutic agent is selected from chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, and endostatin, or a combination thereof.

In some embodiments, the additional therapeutic agent is selected from: nitrogen mustards such as for example, bendamustine, chlorambucil, chlormethine, cyclophosphamide, ifosfamide, melphalan, prednimustine, trofosfamide; alkyl sulfonates like busulfan, mannosulfan, treosulfan; ethylene Imines like carboquone, thiotepa, triaziquone; nitrosoureas like carmustine, fotemustine, lomustine, nimustine, ranimustine, semustine, streptozocin; epoxides such as for example, etoglucid; other alkylating agents such as for example dacarbazine, mitobronitol, pipobroman, temozolomide; folic acid analogues such as for example methotrexate, permetrexed, pralatrexate, raltitrexed; purine analogs such as for example cladribine, clofarabine, fludarabine, mercaptopurine, nelarabine, tioguanine; pyrimidine analogs such as for example azacitidine, capecitabine, carmofur, cytarabine, decitabine, fluorouracil, gemcitabine, tegafur; vinca alkaloids such as for example vinblastine, vincristine, vindesine, vinflunine, vinorelbine; podophyllotoxin derivatives such as for example etoposide, teniposide; colchicine derivatives such as for example demecolcine; taxanes such as for example docetaxel, paclitaxel, paclitaxel poliglumex; other plant alkaloids and natural products such as for example trabectedin; actinomycines such as for example dactinomycin; antracyclines such as for example aclarubicin, daunorubicin, doxorubicin, epirubicin, idarubicin, mitoxantrone, pirarubicin, valrubicin, zorubincin; other cytotoxic Antibiotics such as for example bleomycin, ixabepilone, mitomycin, plicamycin; platinum compounds such as for example carboplatin, cisplatin, oxaliplatin, satraplatin; methylhydrazines such as for example procarbazine; sensitizers such as for example aminolevulinic acid, efaproxiral, methyl aminolevulinate, porfimer sodium, temoporfin; protein kinase inhibitors such as for example dasatinib, erlotinib, everolimus, gefitinib, imatinib, lapatinib, nilotinib, pazonanib, sorafenib, sunitinib, temsirolimus; other antineoplastic agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; estrogens such as for example diethylstilbenol, ethinylestradiol, fosfestrol, polyestradiol phosphate; progestogens such as for example gestonorone, medroxyprogesterone, megestrol; gonadotropin releasing hormone analogs such as for example buserelin, goserelin, leuprorelin, triptorelin; anti-estrogens such as for example fulvestrant, tamoxifen, toremifene; Anti-Androgens such as for example bicalutamide, flutamide, nilutamide, enzyme inhibitors, aminoglutethimide, anastrozole, exemestane, formestane, letrozole, vorozole; other hormone antagonists such as for example abarelix, degarelix; immunostimulants such as for example histamine dihydrochloride, mifamurtide, pidotimod, plerixafor, roquinimex, thymopentin; Immunosuppressants such as for example everolimus, gusperimus, leflunomide, mycophenolic acid, sirolimus; calcineurin inhibitors such as for example ciclosporin, tacrolimus; other immunosuppressants such as for example azathioprine, lenalidomide, methotrexate, thalidomide; and radiopharmaceuticals such as for example, iobenguane.

In some embodiments, the additional therapeutic agent is selected from: interferons, interleukins, tumor necrosis factors, and growth factors, or the like.

In some embodiments, the additional therapeutic agent is selected from: ancestim, filgrastim, lenograstim, molgramostim, pegfilgrastim, sargramostim; Interferons such as for example interferon alfa natural, interferon alfa-2a, interferon alfa-2b, interferon alfacon-1, interferon alfa-n1, interferon beta natural, interferon beta-1a, interferon beta-1b, interferon gamma, peginterferon alfa-2a, peginterferon alfa-2b; Interleukins such as for example aldesleukin, oprelvekin; Other Immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; Immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNF alpha Inhibitors such as for example adalimumab, afelimomab, certolizumab pegol, etanercept, golimumab, infliximab; Interleukin Inhibitors such as for example anakinra, basiliximab, canakinumab, daclizumab, mepolizumab, rilonacept, tocilizumab, ustekinumab; Calcineurin Inhibitors such as for example ciclosporin, tacrolimus; and Other Immunosuppressants such as for example azathioprine, lenalidomide, methotrexate, thalidomide.

In some embodiments, the additional therapeutic agent is selected from: adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, panitumumab, ranibizumab, rituximab, tositumomab, and trastuzumab, or the like, or a combination thereof.

In some embodiments, the additional therapeutic agent is selected from: monoclonal antibodies such as for example alemtuzumab, bevacizumab, catumaxomab, cetuximab, edrecolomab, gemtuzumab, ofatumumab, panitumumab, rituximab, trastuzumab; immunosuppressants, eculizumab, efalizumab, muromab-CD3, natalizumab; TNF alpha inhibitors such as for example adalimumab, afelimomab, certolizumab pegol, golimumab, infliximab; interleukin inhibitors, basiliximab, canakinumab, daclizumab, mepolizumab, tocilizumab, ustekinumab; radiopharmaceuticals, ibritumomab tiuxetan, tositumomab; others monoclonal antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, and zalutumumab.

In some embodiments, the additional therapeutic agent is selected from: agents that affect the tumor micro-environment such as cellular signaling network (e.g., phosphatidylinositol 3-kinase (PI3K) signaling pathway, signaling from the B-cell receptor and the IgE receptor). In some embodiments, the additional therapeutic agent is a PI3K signaling inhibitor or a Syk kinase inhibitor. In one embodiment, the Syk inhibitor is R788. In another embodiment is a PKCγ inhibitor such as by way of example only, enzastaurin.

Examples of agents that affect the tumor micro-environment include PI3K signaling inhibitor, Syk kinase inhibitor, protein kinase inhibitors such as for example dasatinib, erlotinib, everolimus, gefitinib, imatinib, lapatinib, nilotinib, pazonanib, sorafenib, sunitinib, temsirolimus; other angiogenesis inhibitors such as for example GT-111, JI-101, R1530; other kinase inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, BI6727, BIBF 1120, BIBW 2992, BMS-690154, BMS-777607, BMS-863233, BSK-461364, CAL-101, CEP-11981, CYC116, DCC-2036, dinaciclib, dovitinib lactate, E7050, EMD 1214063, ENMD-2076, fostamatinib disodium, GSK2256098, GSK690693, INCB18424, INNO-406, JNJ-26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, R05185426, SAR103168, SCH 727965, SGI-1176, SGX523, SNS-314, TAK-593, TAK-901, TKI258, TLN-232, TTP607, XL147, XL228, XL281R05126766, XL418, and XL765.

In some embodiments, the additional therapeutic agent is selected from: inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

In some embodiments, the additional therapeutic agent is selected from: adriamycin, dactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL2), interferon alfa-2a; interferon alfa-2b; interferon alfa-n1; interferon alfa-n3; interferon beta-1a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In some embodiments, the additional therapeutic agent is selected from: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, the additional therapeutic agent is selected from: alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), or purine analogs (e.g., mercaptopurine, thioguanine, pentostatin).

In some embodiments, the additional therapeutic agent is selected from: nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), and triazenes (decarbazine, ete.). Examples of antimetabolites include, but are not limited to folic acid analog (e.g., methotrexate), or pyrimidine analogs (e.g., fluorouracil, floxouridine, Cytarabine), or purine analogs (e.g., mercaptopurine, thioguanine, pentostatin).

In some embodiments, the additional therapeutic agent is selected from: agents that act by arresting cells in the G2-M phases due to stabilized microtubules, e.g., Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

### Pharmaceutical Compositions and Formulations

Disclosed herein, in certain embodiments, are pharmaceutical compositions and formulations comprising: (a) BTK inhibitor; (b) an mTOR inhibitor or pazopanib or salt thereof, and (c) a pharmaceutically-acceptable excipient. In some embodiments, the BTK inhibitor is ibrutinib. In some embodiments, a pharmaceutical composition comprising: (a) a BTK inhibitor; (b) an mTOR inhibitor; and (c) a pharmaceutically-acceptable excipient, is provided. Exemplary mTOR inhibitors are sirolimus and everolimus, and an exemplary BTK inhibitor is ibrutinib. In some embodiments, the combination is in a combined dosage form. In some embodiments, the combination is in separate dosage forms.

Disclosed herein, in certain embodiments, are pharmaceutical compositions and formulations comprising: (a) a BTK inhibitor; (b) an anti-cancer agent (e.g., paclitaxel; docetaxel; or an EGFR inhibitor); and (c) a pharmaceutically-acceptable excipient. An exemplary BTK inhibitor is ibrutinib. An exemplary EGFR inhibitor is cetuximab. In some embodiments, the combination is in a combined dosage form. In some embodiments, the combination is in separate dosage forms.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference in their entirety.

A pharmaceutical composition, as used herein, refers to a mixture of a compound described herein, such as, for example, ibrutinib and an anticancer agent, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound described herein and a co-agent, are both administered to a patient simultaneously in the form of a single entity or dosage. The term “non-fixed combination” means that the active ingredients, e.g., a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and An anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®) , cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil)(Sterotex®, higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, or ng of therapeutic agent per mL, dL, or L of blood serum, absorbed into the bloodstream after administration. As used herein, measurable plasma concentrations are typically measured in ng/mL or μg/mL.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. In some embodiments, the composition is formulated for administration in a combined dosage form. In some embodiments, the composition is formulated for administration in a separate dosage forms. As used herein, the term “subject” is used to mean an animal, preferably a mammal, including a human or non-human. The terms “individual(s)”, “subject(s)” and “patient(s)” are used interchangeably herein, and mean any mammal. In some embodiments, the mammal is a human. In some embodiments, the mammal is a non-human. None of the terms require or are limited to situations characterized by the supervision (e.g., constant or intermittent) of a health care worker (e.g., a doctor, a registered nurse, a nurse practitioner, a physician's assistant, an orderly or a hospice worker).

Moreover, the pharmaceutical compositions described herein, which include ibrutinib and/or an anticancer agent can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations described herein may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of ibrutinib and/or an anticancer agent, with one or more pharmaceutical excipients to form a bulk blend composition. When referring to these bulk blend compositions as homogeneous, it is meant that the particles of ibrutinib and/or an anticancer agent, are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of ibrutinib and/or an anticancer agent. In another embodiment, some or all of the particles of ibrutinib and/or an anticancer agent, are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of ibrutinib and/or an anticancer agent, from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g., Hypromellose USP Pharmacoat-603, hydroxypropylmethylcellulose acetate stearate (Aqoate HS-LF and HS), hydroxyethylcellulose, hydroxypropylcellulose (e.g., Klucel), ethylcellulose (e.g., Ethocel®), and microcrystalline cellulose (e.g., Avicel®) , microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g., Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized.

Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of ibrutinib or the second agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of ibrutinib or the second agent, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of ibrutinib and/or an anticancer agent, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with ibrutinib and/or an anticancer agent, which sufficiently isolate the compound of any of ibrutinib or an anticancer agent, from other non-compatible excipients. Materials compatible with compounds of any of ibrutinib or an anticancer agent, are those that delay the release of the compounds of any of ibrutinib or an anticancer agent, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds of any of ibrutinib or an anticancer agent may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of compounds of any of ibrutinib or an anticancer agent are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds of any of ibrutinib and/or an anticancer agent are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound of any of ibrutinib and/or an anticancer agent, described herein, may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles<1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include ibrutinib and/or an anticancer agent, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of ibrutinib and/or an anticancer agent, described herein and at least one dispersing agent or suspending agent for oral administration to a subject. The formulations may be a powder and/or granules for suspension, and upon admixture with water, a substantially uniform suspension is obtained. Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g., HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g., HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated by reference. Formulations that include ibrutinib and/or An anticancer agent, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation described herein may be in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with ibrutinib and/or An anticancer agent, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In one embodiment, the transdermal formulations described herein include at least three components: (1) a formulation of a compound of ibrutinib and an anticancer agent; (2) a penetration enhancer; and (3) an aqueous adjuvant. In addition, transdermal formulations can include additional components such as, but not limited to, gelling agents, creams and ointment bases, and the like.

In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of ibrutinib and An anticancer agent. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include a compound of ibrutinib and/or an anticancer agent, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Other Formulations

In certain embodiments, delivery systems for pharmaceutical compounds may be employed, such as, for example, liposomes and emulsions. In certain embodiments, compositions provided herein can also include an mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Dosing and Treatment Regimens

In some embodiments, the amount of ibrutinib that is administered in combination with an anticancer agent is from about 10 mg/day up to, and including, about 1000 mg/day. In some embodiments, the amount of ibrutinib that is administered is from about 40 mg/day to 70 mg/day. In some embodiments, the amount of ibrutinib that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the amount of ibrutinib that is administered is about 40 mg/day. In some embodiments, the amount of ibrutinib that is administered is about 50 mg/day. In some embodiments, the amount of ibrutinib that is administered is about 60 mg/day. In some embodiments, the amount of ibrutinib that is administered is about 70 mg/day. In some embodiments, the amount of ibrutinib that is administered per day is about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 700 mg, or about 840 mg. In some embodiments, the amount of ibrutinib that is administered per day is less than about 10 mg, or greater than about 1000 mg. In some embodiments, ibrutinib is not administered every day, i.e., it may be administered every other day or intermittently.

In some embodiments, the amount of pazopanib (or a salt thereof) that is administered daily with ibrutinib is from about 1 mg to about 100 mg; about 200 mg to about 800 mg, about 400 mg to about 800 mg, or about 600 mg to about 800 mg. In some embodiments, the daily dose of pazopanib is about 200 mg to about 800 mg. In some embodiments, the daily dose of pazopanib is about 400 mg to about 800 mg. In some embodiments, the daily dose of pazopanib is about 600 mg to about 800 mg. In some embodiments, the daily dose of pazopanib is greater than about 800 mg.

In some embodiments, pazopanib or a salt of pazopanib is administered once per day, twice per day, three times per day, or four times per day. In some embodiments, pazopanib or a salt of pazopanib is administered once per day. In some embodiments, pazopanib or a salt of pazopanib is administered twice per day. In some embodiments, pazopanib or a salt of pazopanib is administered three times per day. In some embodiments, pazopanib or a salt of pazopanib is administered four times per day. In some embodiments, pazopanib or a salt of pazopanib is not administered daily. In some embodiments, pazopanib or a salt of pazopanib may be administered on a particular day(s) even if BTK inhibitor such as ibrutinib is not administered on that particular day(s), i.e., during an ibrutinib drug holiday. In some embodiments, the pazopanib or a salt thereof is not administered daily.

In some embodiments, the amount of an mTOR inhibitor (i.e., everolimus) that is administered daily in combination with ibrutinib is from about 1 mg to about 50 mg; from about 1.5 mg to about 25 mg; from about 2.0 to about 20 mg; from about 2.5 to about 15 mg; from about 3.0 to about 10 mg; or from about 5.0 mg to about 7.5 mg. In some embodiments, the amount of mTOR inhibitor that is administered daily in combination with ibrutinib is about 2.0 mg; about 2.5 mg; about 3.0 mg; about 3.5 mg; about 3.5 mg; about 4.0 mg; about 4.5 mg; about 5.0 mg; about 5.5 mg; about 6.0 mg; about 6.5 mg; about 7.0 mg; about 7.5 mg; about 8.0 mg; about 8.5 mg; about 9.0 mg; about 9.5 mg; about 10.0 mg. In some embodiments, the amount of mTOR inhibitor is less than about 1 mg or greater than about 10 mg. In some embodiments, the mTOR inhibitor (i.e., everolimus) may be administered on a particular day(s) even if BTK inhibitor such as ibrutinib is not administered on that particular day(s), i.e., during an ibrutinib drug holiday. In some embodiments, the mTOR inhibitor is not administered daily. In some embodiments, the amount of mTOR inhibitor (i.e., everolimus) that is administered is about 10 mg per day. In some embodiments, the mTOR inhibitor (i.e., everolimus) is administered orally.

In some embodiments, the amount of an mTOR inhibitor (i.e., sirolimus) that is administered daily in combination with ibrutinib is from about 1 mg to about 50 mg; from about 1.5 mg to about 25 mg; from about 2.0 to about 20 mg; from about 2.5 to about 15 mg; from about 3.0 to about 10 mg; from about 5.0 mg to about 7.5 mg. In some embodiments, the amount of mTOR inhibitor that is administered daily in combination with ibrutinib is about 2.0 mg; about 2.5 mg; about 3.0 mg; about 3.5 mg; about 3.5 mg; about 4.0 mg; about 4.5 mg; about 5.0 mg; about 5.5 mg; about 6.0 mg; about 6.5 mg; about 7.0 mg; about 7.5 mg; about 8.0 mg; about 8.5 mg; about 9.0 mg; about 9.5 mg; about 10.0 mg. In some embodiments, the amount of mTOR inhibitor is less than about 1 mg or greater than about 10 mg. In some embodiments, the mTOR inhibitor (i.e., sirolimus) may be administered on a particular day(s) even if BTK inhibitor such as ibrutinib is not administered on that particular day(s), i.e., during an ibrutinib drug holiday. In some embodiments, the mTOR inhibitor is not administered daily. In some embodiments, the mTOR inhibitor is not administered daily. In some embodiments, the amount of mTOR inhibitor (i.e., sirolimus) that is administered is about 10 mg per day. In some embodiments, the mTOR inhibitor (i.e., sirolimus) is administered orally.

In some embodiments, the amount of paclitaxel that is administered is about 40 mg/m² to about 120 mg/m² per week. In some embodiments, the amount of paclitaxel that is administered is about 60 mg/m² to about 100 mg/m² per week. In some embodiments, the amount of paclitaxel that is administered is about 80 mg/m² per week. The paclitaxel may be administered intravenously. The weekly paclitaxel dosage may be administered at one time or at multiple times during the week. In some embodiments, the amount of paclitaxel that is administered is less than about 40 mg/m² or greater than about 120 mg/m² per week. In some embodiments, the paclitaxel is not administered weekly, e.g., is administered every other week or on an as-needed basis. In some embodiments, paclitaxel is administered intravenously. In some embodiments, paclitaxel is not administered. For example, a suitable replacement for paclitaxel may be administered, e.g., another suitable taxane may be administered.

In some embodiments, the amount of docetaxel that is administered is from about 25 mg/m² to about 125 mg/m² every three weeks. In some embodiments, the amount of docetaxel that is administered is about 50 mg/m² to about 100 mg/m² every three weeks. In some embodiments, the amount of paclitaxel that is administered is about 75 mg/m² every three weeks. The docetaxel may be administered intravenously. The docetaxel dosage may be administered at one time or at multiple times during the week that it is administered. In some embodiments, the amount of docetaxel that is administered every three weeks is less than about 25 mg/m² or greater than about 125 mg/m². In some embodiments, the docetaxel is not administered every three weeks, e.g., is administered every other week, on an as-needed basis, or intermittently. In some embodiments, docetaxel is administered intravenously. In some embodiments, docetaxel is not administered. For example, a suitable replacement for docetaxel may be administered, e.g., another suitable taxane may be administered in lieu of, or in combination with, docetaxel.

In some embodiments, cetuximab is administered in two different dosage amounts. In this regard, in some embodiments, the initial dosage of cetuximab that is administered is different from the subsequent dosages of cetuximab that is administered. This initial dosage of cetuximab may be administered only once during the treatment and/or only once during each cycle. Each dosage after the initial dosage is at the subsequent dosage. In some embodiments, the initial dosage of cetuximab that is administered is from about 200 mg/m² to about 600 mg/m². In some embodiments, the initial dosage of cetuximab that is administered is about 400 mg/m². In some embodiments, the initial dosage of cetuximab that is administered is less than about 200 mg/m² or greater than about 600 mg/m². In some embodiments, the initial dosage of cetuximab is administered intravenously. In some embodiments, each subsequent dosage of cetuximab that is administered is from about 100 mg/m² to about 400 mg/m² per week. In some embodiments, each subsequent dosage of cetuximab that is administered is about 250 mg/m² per week. In some embodiments, each subsequent dosage of cetuximab that is administered is less than about 100 mg/m² or greater than about 400 mg/m² per week. In some embodiments, the subsequent dosage(s) of cetuximab are administered intravenously. In some embodiments, the subsequent dosage(s) of cetuximab that are administered are the same as the initial dosage of cetuximab that is administered. Cetuximab may be administered once per week, multiple times in one week, once every two weeks, as needed, intermittently, and the like. In some embodiments, cetuximab is administered intravenously. In some embodiments, cetuximab is not administered. For example, a suitable replacement for cetuximab may be administered, e.g., another suitable EGFR inhibitor may be administered.

In some embodiments, the dosing regimen is followed in cycles. In some embodiments, each cycle is 21 days. In some embodiments, each cycle is less than 21 days or greater than 21 days. For example, each cycle may be 14 days, 15 days, 16 days, 17 days, 18 days, 19 days, 20 days, 22 days, 23 days, 24 days, 25 days, 26 days, 27 days, 28 days, and so forth.

In some embodiments, the dosing regimen is followed for any number of cycles. In some embodiments, the dosing regimen may be followed for at least 1, 2, 3, 4, 5, 6, 7, 8, 9, 10, 11, or 12 cycles. In some embodiments, the dosing regimen is followed for more than 12 cycles.

In some embodiments, a dosing regimen described herein is administered to the subject over a period of time of up to 5 years, 4 years, 3 years, 2 years, or 1 year. In some instances, the combination dosing regime is administered for a period of up to 40 cycles, 35 cycles, 30 cycles, 25 cycles, 20 cycles, 15 cycles, 14 cycles, 13 cycles, 12 cycles, 11 cycles, or 10 cycles. In some instances, the dosing regimen is administered for a period of up to 20 cycles. In some instances, the dosing regimen is administered for a period of up to 15 cycles. In some instances, the dosing regimen is administered for a period of up to 13 cycles. In some instances, the dosing regimen is administered for a period of up to 12 cycles.

In some embodiments, the solid tumor is relapsed and/or refractory. In some embodiments, the subject has received at least one prior therapy. In some embodiments, the subject has received at least two prior therapies. In some embodiments, the prior therapy comprises VEGF-TKI. In some embodiments, the prior therapy comprises cisplatin. In some embodiments, the prior therapy comprises a fluoropyrimidine regimen. In some embodiments, the prior therapy comprises an irinotecan and an oxaliplatin-based regimen. In some embodiments, the subject is unable to tolerate irinotecan chemotherapy.

In some embodiments, the subject having renal cell carcinoma has not been treated with everolimus or temsirolimus. In some embodiments, the subject having urothelial carcinoma or gastric adenocarcinoma has not been treated with a taxane. In some embodiments, the subject having colorectal cancer or cancer has not been treated with cetuximab or pantimumab.

In some embodiments, the compositions disclosed herein are administered for prophylactic, therapeutic, or maintenance treatment. In some embodiments, the compositions disclosed herein are administered for therapeutic applications. In some embodiments, the compositions disclosed herein are administered for therapeutic applications. In some embodiments, the compositions disclosed herein are administered as a maintenance therapy, for example for a patient in remission.

In the case wherein the patient's status does improve, upon the doctor's discretion the administration of the compounds may be given continuously; alternatively, the dose of drug (i.e., the BTK inhibitor, mTOR inhibitor, pazopanib, paclitaxel, docetaxel, and/or cetuximab) being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In certain embodiments, the invention relates to any of the pharmaceutical compositions or methods described herein, wherein the pharmaceutical composition or method comprises ibrutinib or its use; and the unit dosage of ibrutinib is a capsule comprising 140 mg of ibrutinib.

In certain embodiments, the invention relates to any of the pharmaceutical compositions or methods described herein, wherein the pharmaceutical composition or method comprises everolimus or its use; and the unit dosage of everolimus is a tablet comprising 2.5 mg, 5 mg, 7.5 mg, or 10 mg of everolimus. In certain embodiments, the invention relates to any of the pharmaceutical compositions or methods described herein, wherein the pharmaceutical composition or method comprises everolimus or its use; and the unit dosage of everolimus is a tablet comprising 10 mg of everolimus.

In certain embodiments, the invention relates to any of the pharmaceutical compositions or methods described herein, wherein the pharmaceutical composition or method comprises paclitaxel or its use; the unit dosage of paclitaxel is a vial comprising 5 mL, 16.7 mL, or 50 mL of a paclitaxel solution; and the paclitaxel solution comprises 6 mg/mL of paclitaxel. In certain embodiments, the paclitaxel solution further comprises 527 mg/mL of purified polyoxyl 35 castor oil and 49.7% (v/v) dehydrated alcohol, USP.

In certain embodiments, the invention relates to any of the pharmaceutical compositions or methods described herein, wherein the pharmaceutical composition or method comprises docetaxel or its use; the unit dosage of docetaxel is a vial comprising 1 mL or 4 mL of a docetaxel solution; and the docetaxel solution comprises 20 mg/mL of docetaxel. In certain embodiments, the docetaxel solution further comprises 50/50 (v/v) ratio polysorbate 80/dehydrated alcohol.

In certain embodiments, the invention relates to any of the pharmaceutical compositions or methods described herein, wherein the pharmaceutical composition or method comprises cetuximab or its use; the unit dosage of cetuximab is a vial comprising 50 mL or 100 mL of a cetuximab solution; and the cetuximab solution comprises 2 mg/mL of cetuximab. In certain embodiments, the cetuximab solution further comprises 8.48 mg/mL sodium chloride, 1.88 mg/mL sodium phosphate dibasic heptahydrate, 0.41 mg/mL sodium phosphate monobasic monohydrate, and Water for Injection, USP.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials.

Examples of pharmaceutical packaging materials include, but are not limited to, blister packs, bottles, tubes, bags, containers, bottles, and any packaging material suitable for a selected formulation and intended mode of administration and treatment.

For example, the container(s) include ibrutinib, optionally in a composition or in combination with an anticancer agent such as an mTOR inhibitor; pazopanib; paclitaxel; docetaxel; or cetuximab as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1. Ibrutinib in Combination with mTOR Inhibitor Sirolimus in a Syngeneic RCC Model

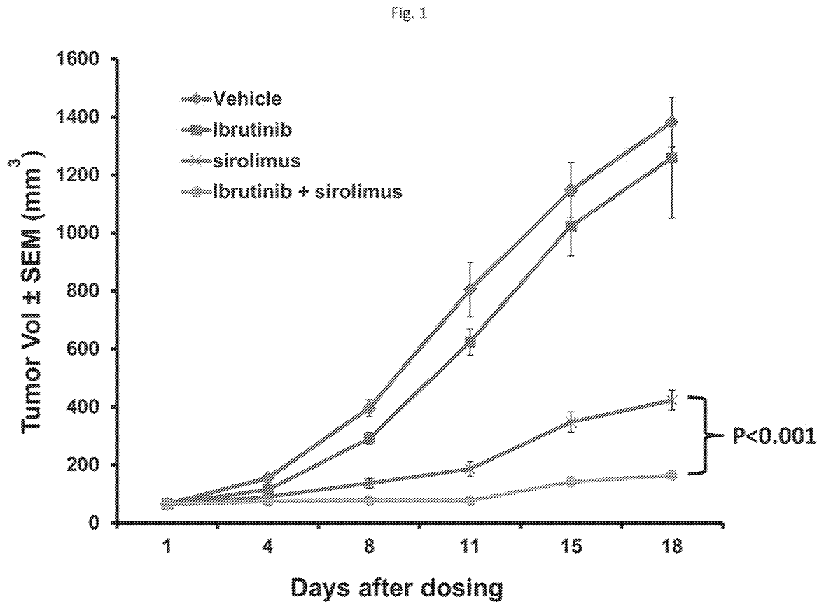

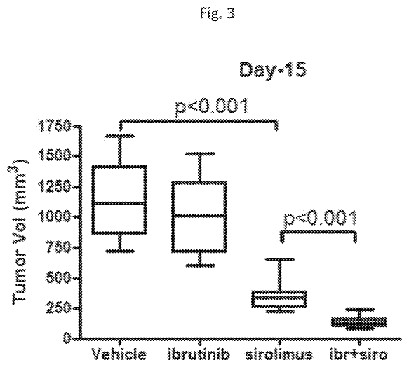

The syngeneic RCC model Renca was used for this experiment. As shown in FIG. 1, the combination of ibrutinib and sirolimus resulted in reduced tumor volume in the model as compared to what would have been expected based on the additive effect of ibrutinib alone and sirolimus alone. Tumor volume on day 15 is shown in FIG. 3.

### Example 2. Ibrutinib in Combination with mTOR Inhibitor Everolimus in a Xenograft RCC Model

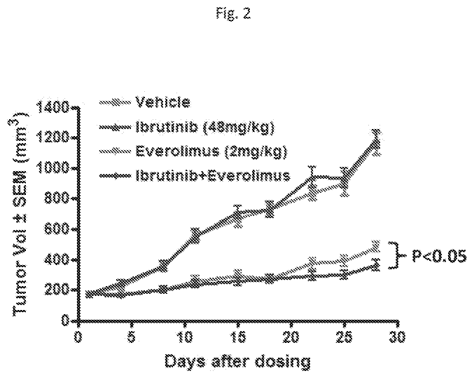

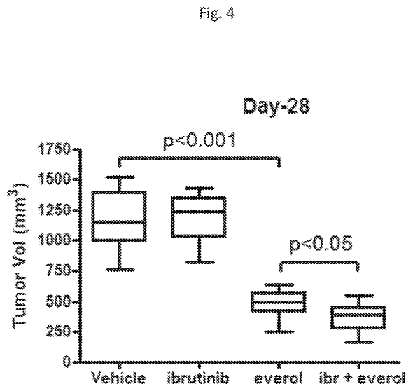

The human renal cancer cell line 786-0 (RCC cells) was used for this in vivo experiment. RCC cells were subcutaneously implanted into SCID mice. These mice were separated into (4) groups. Group 1 was a vehicle control group. Group 2 was administered 48 mg/kg of ibrutinib. Group 3 was administered 2 mg/kg of everolimus. Group 4 was administered a combination of 48 mg/kg of ibrutinib and 2 mg/kg of everolimus. As shown in FIG. 2, the combination of ibrutinib and everolimus resulted in reduced tumor volume as compared to what would have been expected based on the additive effect of ibrutinib alone and everolimus alone. Tumor volume on Day 28 is shown in FIG. 4.

### Example 3. Ibrutinib in Combination with mTOR Inhibitors in Renal Cell Carcinoma In Vitro

Cells and Reagents.

Cell lines 769-P, 786-0, A498, ACHN, Caki-1, and RENCA were obtained from American Type Culture Collection (ATCC) and cultured as recommended. Antibodies to EGFR, pEGFR (Y1086), HER2, Akt, pAkt (S473); pAkt (T308), mTOR, p-mTOR, S6, pS6, ERK, pERK, MET, pMET, and pBtk (Y223) were obtained from Cell Signaling Technology. Antibodies to α-tubulin were obtained from Santa Cruz Biotechnology, Inc. As is known, pAkt (T308) refers to the Akt protein phosphorylated at amino acid residue T308, and pAkt (S473) refers to the Akt protein phosphorylated at amino acid residue S473.

Cell Proliferation Assay:

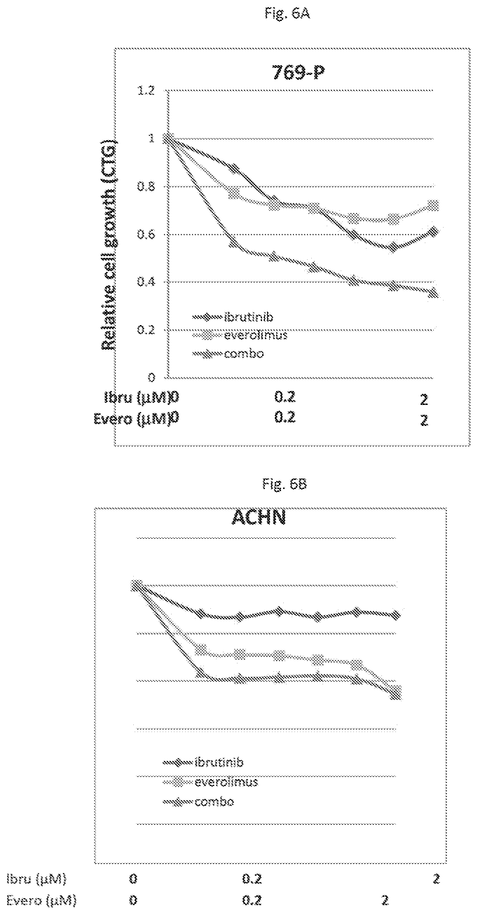

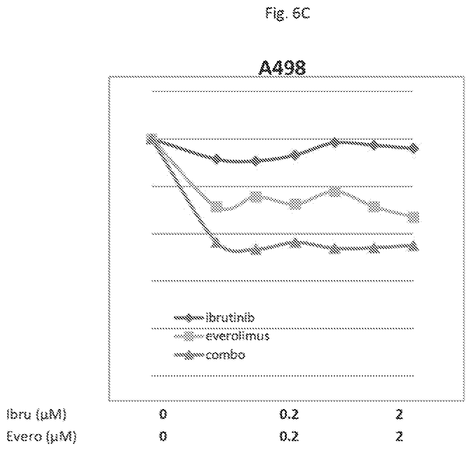

The CellTiter-Glo® Luminescent Cell Viability assay was performed according to manufacturer's instructions. Briefly, cells were seeded at 8,000-10,000 cells/well in a 96-well plate overnight before adding ibrutinib or mTOR inhibitor everolimus. Ibrutinib and everolimus were added in combination for 72 hours. The number of viable cells in culture was determined by the quantification of ATP present, which was proportional to the luminal signal detected. As shown in FIGS. 6A-6C, ibrutinib potentiated the effect of everolimus on cell growth inhibition in renal cancer cell lines 769-P, ACHN, and A498.

Western Blots:

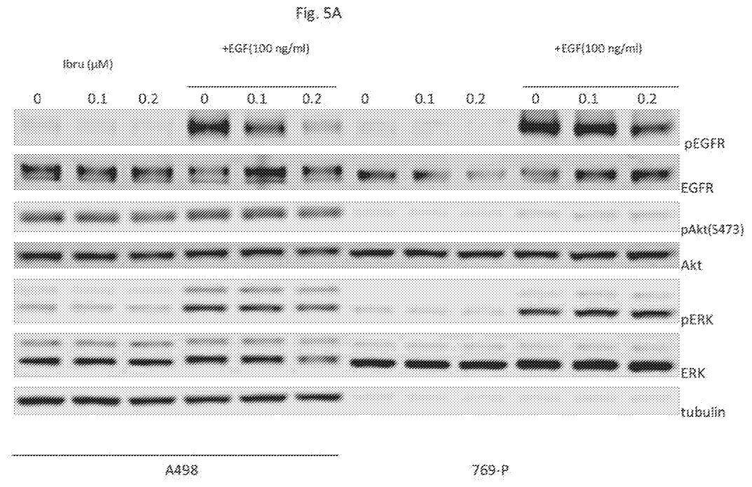

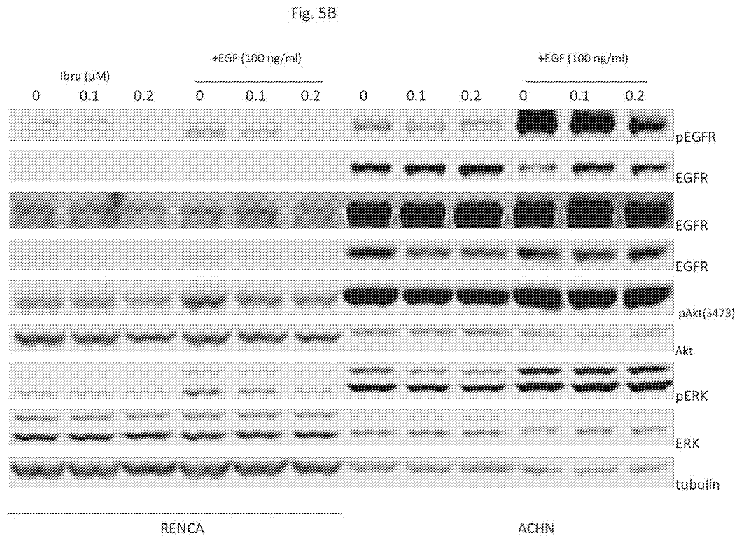

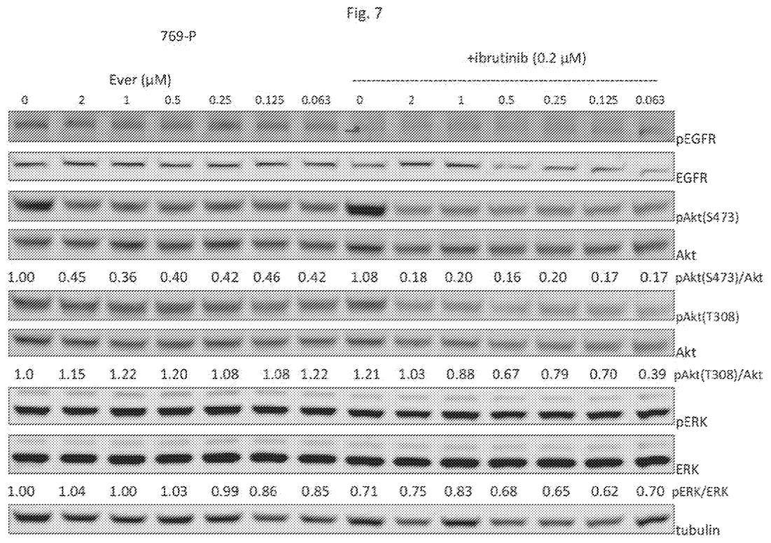

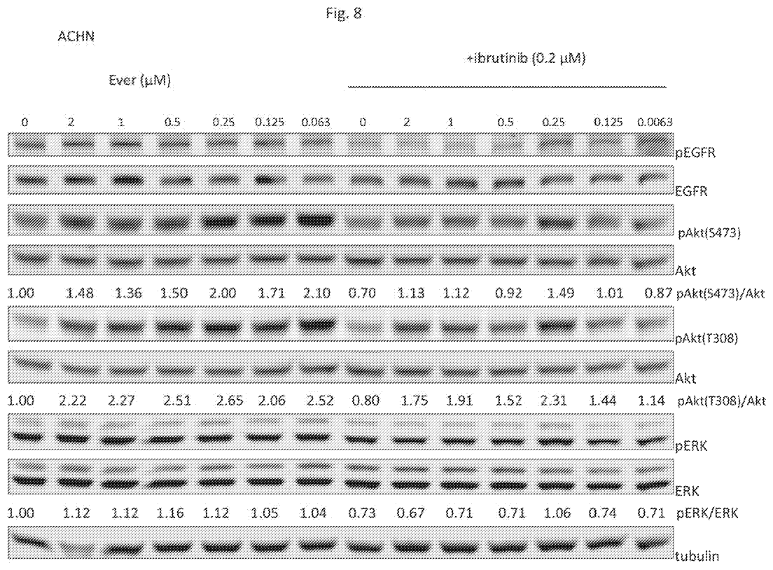

Whole cell lysates in 1× sample buffer (Invitrogen) were electrophoresed on a 4%-12% Bis-Tris gel. After transferring the proteins onto a PVDF membrane, the blot was probed by antibodies, and the signal was detected using the Odyssey imager (LI-COR Biosciences). Paired mouse and rabbit antibodies were used to probe the total protein, and the corresponding phosphorylated proteins. The effect of ibrutinib treatment, with and without EGF stimulation/induction, on various proteins was studied. A one-hour pre-treatment with ibrutinib and a 10-minute stimulation with EGF was conducted. As shown in FIGS. 5A-5B, ibrutinib inhibits EGF-induced pEGFR while demonstrating limited impacts on pAkt and pERK in renal cancer cell lines A498, 769-P, RENCA, and ACHN. As shown in FIG. 7, ibrutinib, when combined with mTOR inhibitor everolimus, further inhibited pAkt (both pAkt T308 and pAkt S473) and pERK in the 769-P renal cell carcinoma cell line. As shown in FIG. 8, everolimus induced up-regulation of pAkt after 24 h treatment; addition of ibrutinib counteracted the up-regulation of pAkt (both pAkt T308 and pAkt S473) by everolimus; and inhibited pERK in the ACHN renal carcinoma cell line. As such, ibrutinib ameliorates the effect (i.e., the undesired effects) of mTOR inhibitor alone.

### Example 4. Effect of the Combination of Ibrutinib and Pazopanib on Cell Growth Inhibition and Apoptosis in Renal Cancer Cell Lines

Cells and Reagents:

Cell lines 769-P, A498, Caki-1, and ACHN were obtained from American Type Culture Collection (ATCC) and cultured as recommended.

Cell Proliferation Assay:

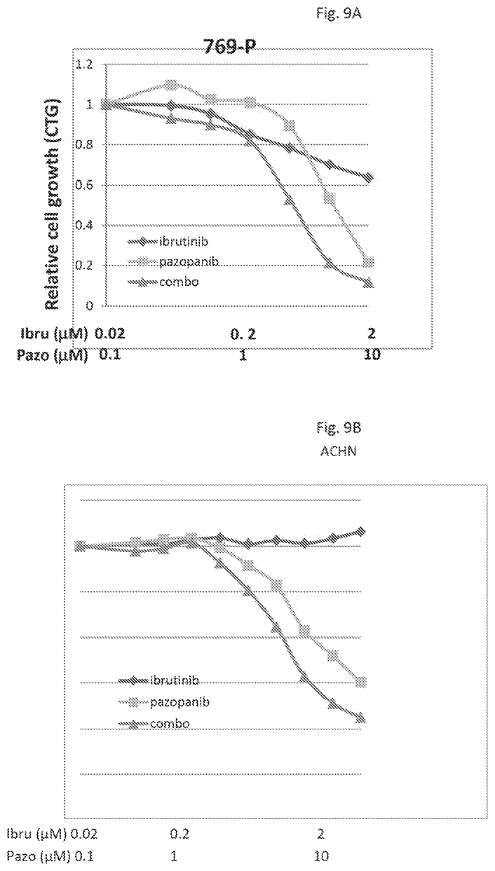

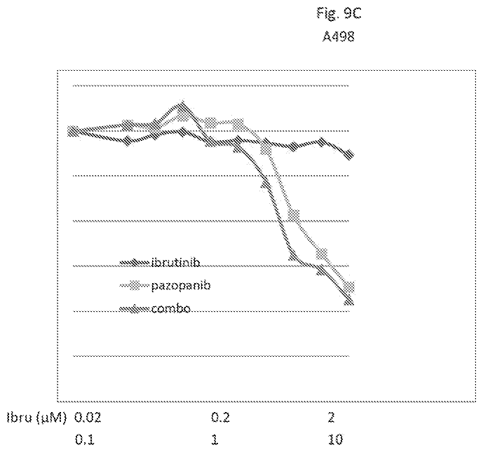

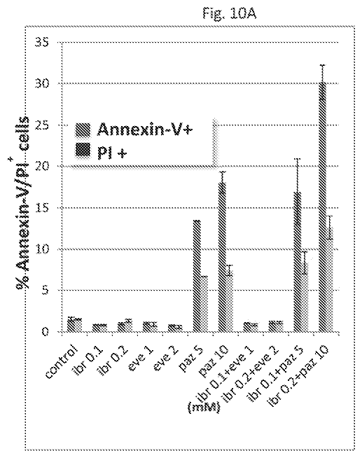

The CellTiter-Glo® Luminescent Cell Viability assay was performed according to manufacturer's instructions. Briefly, cells were seeded at 8,000-10,000 cells/well in a 96-well plate overnight before adding ibrutinib or pazopanib. Ibrutinib and pazopanib were added in combination for 72 hours. The number of viable cells in culture was determined by the quantification of ATP present, which was proportional to the luminal signal detected. As shown in FIGS. 9A-9C, the combination of ibrutinib and pazopanib resulted in increased cell growth inhibition in renal cancer cell lines 769-P, ACHN, and A498. Similar data were obtained for Caki-1 cells.

Apoptosis Assay:

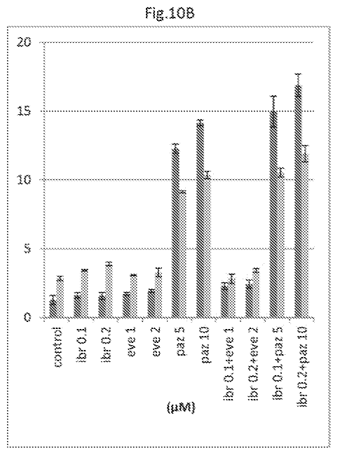

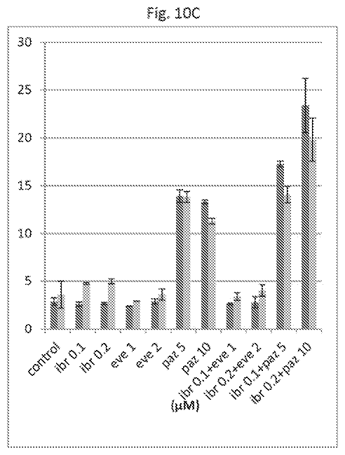

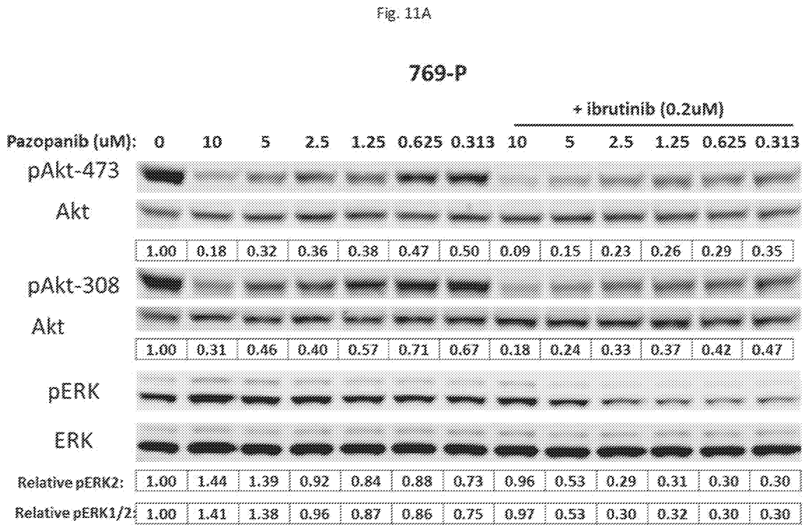

Cells were stained with annexin-V/PI or PI/RNase, and apoptotic cells were quantitated using a FACSCalibur flow cytometer (Becton Dickinson). The number of annexin-V positive cells or subGO cells was calculated. As shown in FIGS. 10A-10C, the combination of ibrutinib and pazopanib resulted in increased apoptosis in renal cancer cell lines 769-P, ACHN, and A498. Similar data were obtained for Caki-1 cells.

Western Blot:

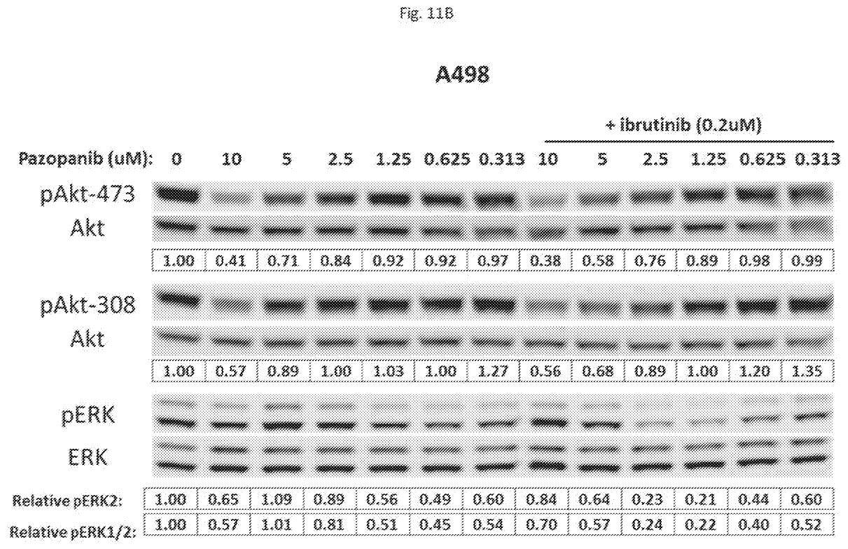

Cell lysates were prepared from RCC cells which were treated with different concentrations of pazopanib for overnight incubation. Antibodies that correlate to the proteins of interest were used for detection. As shown in FIG. 11A-11C, then combined with pazopanib, ibrutinib enhanced the inhibitory effect of pazopanib on pAkt and pErk.

### Example 5. Tumor Growth Inhibition after Administration of a Combination of Ibrutinib and mTOR Inhibitor Everolimus in a 786-0 Xenograft and RENCA Syngeneic Models

Xenograft and Syngeneic Mouse Tumor Models:

786-0 cells were implanted subcutaneously into BALB/c nude mice, and RENCA cells were implanted subcutaneously into BALB/c mice. Treatment with vehicle, ibrutinib, everolimus, and the combination of ibrutinib and everolimus began when 786-0 tumors reached ˜170 mm³, and RENCA tumors reached ˜65 mm³. Ibrutinib and/or everolimus were orally administered once daily at the following dosages: 786-0 xenograft: ibrutinib (48 mg/kg) and everolimus (2, 1, and 0.5 mg/kg), and RENCA syngeneic model: ibrutinib (24 mg/kg) and everolimus (0.3 mg/kg). Tumors were measured twice/week with a vernier caliper and the volume was determined using the formula width×length×0.5. As shown in FIGS. 12A-12B, ibrutinib enhanced the effect of everolimus on tumor growth inhibition in a 786-0 xenograft and a RENCA syngeneic model. Similar results were obtained for the combination of ibrutinib with sirolimus on the RENCA model (FIG. 1).

As shown in FIGS. 1-12B, the combination of ibrutinib and everolimus, sirolimus or pazopanib in renal cancer cell lines (irrespective of VHL mutational status) or animal models shows enhanced effect. Ibrutinib can inhibit, and/or reduce the expression of, EGFR. Additionally, ibrutinib can counteract everolimus-induced up-regulation of pAkt. Ibrutinib may also enhance the inhibition of pAkt and/or pERK.

### Example 6. Tumor Growth Inhibition after Administration of a Combination of Ibrutinib and EGFR Inhibitor Cetuximab in FaDu Human Head and Neck Xenografts

Cells and Reagents:

Cell line FaDu was obtained from American Type Culture Collection (ATCC) and cultured as recommended.

Xenograft Mouse Tumor Model:

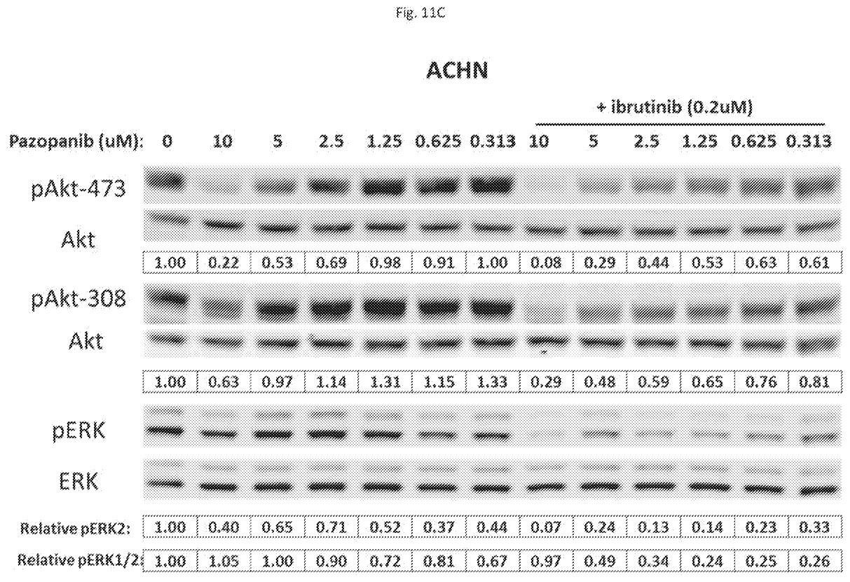

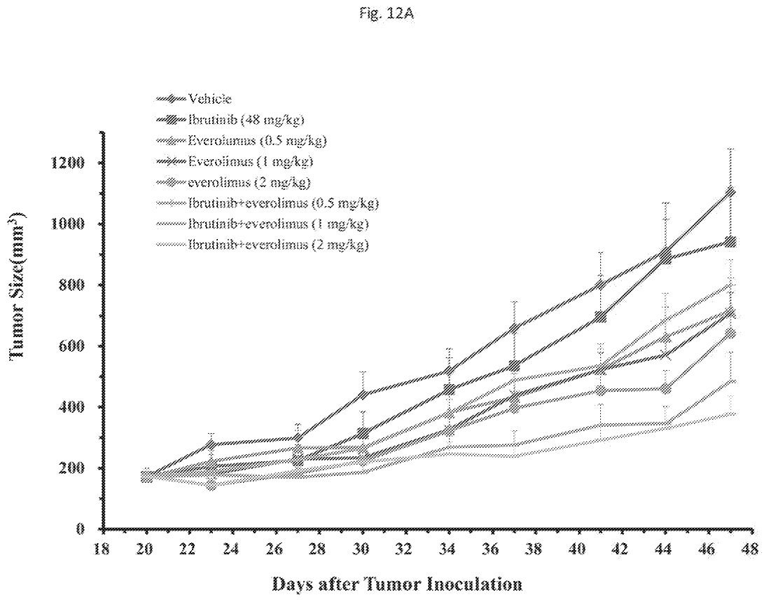

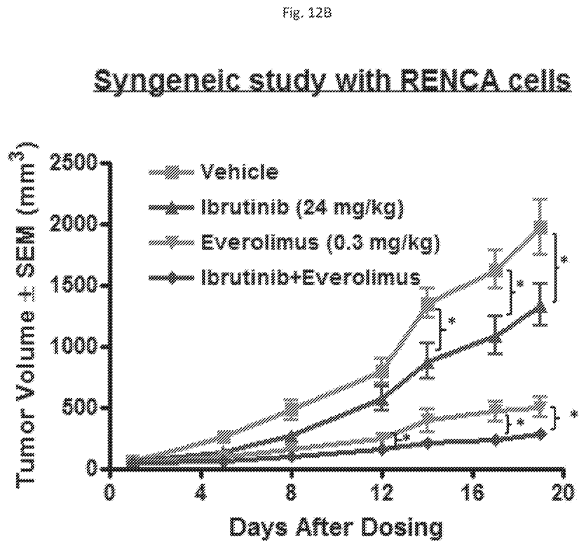

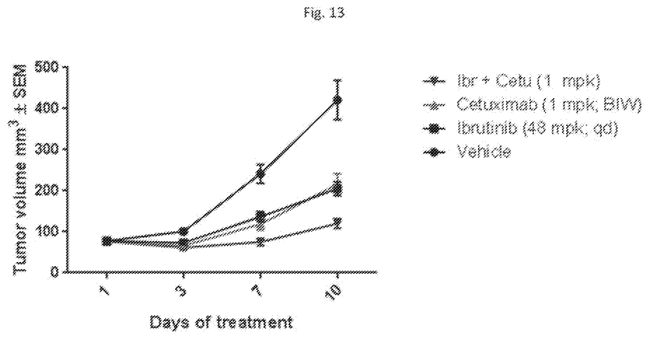

FaDu cells were implanted subcutaneously into BALB/c nude mice at about 5×10⁶ cells/uL. When tumor size reached approximately 120 mm², the mice were treated with vehicle, ibrutinib, cetuximab, or the combination of ibrutinib and cetuximab at the following dosages: (1) ibrutinib only: 48 mg/kg once a day; (2) cetuximab only: 1 mg/kg, twice a week; (2) combination of ibrutinib and cetuximab: ibrutinib 48 mg/kg once a day, and cetuximab 1 mg/kg twice a week. As is shown in FIG. 13, tumor growth inhibition in the FaDu xenograft was enhanced by the combination.

### Example 7

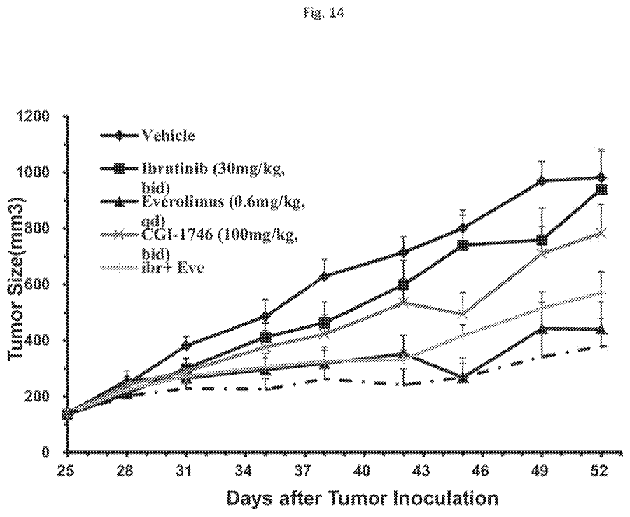

The xenograft model is similar to that described in Examples 2 and 5. Ibrutinib and CGI-1746 were dosed b.i.d. at 30 and 100 mg/kg respectively for each dose alone or in combination with everolimus (0.6 mg/kg, qd). Inhibition of tumor growth is shown in FIG. 14.

### Example 8. A Phase 1b/2 Study of Ibrutinib Combination Therapy in Selected Advanced Gastrointestinal and Genitourinary Tumors

Indications:

Previously treated metastatic renal cell carcinoma (RCC), advanced urothelial carcinoma, advanced gastric (including gastro-esophageal [GEJ]) adenocarcinoma, and metastatic colorectal adenocarcinoma (CRC).

Therapy:

Ibrutinib will be supplied as 140 mg hard gelatin capsules for oral (PO) administration. Everolimus will be supplied as 5 mg, or 10 mg elongated tablets for oral (PO) administration. Docetaxel will be supplied as a liquid concentrate to produce a final concentration of 0.3 to 0.74 mg/mL to be diluted for intravenous (IV) administration.

Paclitaxel will be supplied as a liquid concentrate to produce a final concentration of 0.3-1.2 mg/mL for intravenous (IV) administration. Cetuximab will be supplied as 100 mg/50 mL or 200 mg/100 mL vials for intravenous (IV) administration.

### Objectives:

Phase 1b:

Primary Objective:

To determine the recommended Phase 2 dose (RP2D) of ibrutinib in combination with everolimus in RCC, paclitaxel in urothelial carcinoma, docetaxel in gastric adenocarcinoma and cetuximab in CRC.

Secondary objectives:
To assess the overall response rate (ORR) of ibrutinib combination therapy in each cohort
To assess the safety and tolerability of ibrutinib combination therapy in each cohort
To assess the disease control rate (DCR) of ibrutinib combination therapy in each cohort
To evaluate the pharmacokinetics (PK) of ibrutinib combination therapy in each cohort

Phase 2:

Primary Objectives:
To assess progression-free survival (PFS) of ibrutinib combination therapy in RCC and urothelial carcinoma
To assess the ORR of ibrutinib combination therapy in gastric adenocarcinoma and CRC

Secondary Objectives:
To assess the PFS of ibrutinib combination therapy in gastric adenocarcinoma and CRC
To assess the ORR of ibrutinib combination therapy in RCC and urothelial carcinoma
To assess the DCR of ibrutinib combination therapy in each cohort
To assess the median overall survival (OS) of ibrutinib combination therapy in each cohort
To assess the safety and tolerability of ibrutinib combination therapy in each cohort

Exploratory Objectives:
Biomarker analysis for response and resistance to ibrutinib based therapy
To assess ITK occupancy during ibrutinib treatment in each cohort
To evaluate the pharmacokinetics (PK) of ibrutinib combination therapy in each cohort

Study Design:

This is an open label, Phase 1b/2 multi-center study to assess the safety and efficacy of ibrutinib combination therapy in subjects with previously treated RCC, urothelial carcinoma, gastric adenocarcinoma, and CRC. Each cohort in this study will assess a different malignancy and anticancer agent in combination and follow an independent and parallel design.

The study will consist of an initial Phase 1b portion primarily to assess the safety of ibrutinib, in combination with each anticancer agent, in order to determine the RP2D for each cohort.

A subsequent Phase 2 portion will assess primary endpoints of PFS (with an incorporated interim analysis) for the genitourinary (GU) malignancies (RCC and urothelial carcinoma) and ORR using a Simon's minimax 2-stage design for the gastrointestinal (GI) malignancies (gastric adenocarcinoma and CRC).

### Phase 1b

The Phase 1b portion of this study is performed independently in four separate cohorts defined by the clinical indication; RCC, urothelial carcinoma, gastric adenocarcinoma, and CRC. Safety and dose limiting toxicity (DLT) assessment will be evaluated in 3-9 subjects at each dose level in a 3+3+3 design. At each dose level, DLT assessment will be performed in the first 3 subjects. If 1 of 3 subjects experience a DLT during the first treatment cycle, the same dose level will be expanded to 6 subjects, and if 2 of the 6 experience a DLT, the same dose level will be expanded to 9 subjects. At the 560 mg/day dose level (DL 1), if 0 out of 3, 1 out of 6, or 2 out of 9 subjects (≤22%) experience a DLT during the first treatment cycle, dose escalation to 840 mg/day will occur. At DL 1 (560 mg/day), if ≥33% of subjects experience a DLT (eg, >2 out of 6 or >2 out of 9 subjects), the dose will be de-escalated to 420 mg/day (dose level minus one; DL −1). At the 840 mg/day dose level (DL 2) cohort, subjects will be enrolled in a similar fashion.

The RP2D will be determined when 6-9 subjects complete the DLT observation period based on the totality of the data including dose reductions (of both ibrutinib and the combination therapy), treatment-limiting toxicities (outside of DLTs), the available pharmacokinetic data and the toxicity profile obtained during Phase 1b. In order to determine the RP2D dose level, a minimum of 6 DLT evaluable subjects will be required at the RP2D dose level who are defined to have completed at least 21 days of treatment with ibrutinib in combination with the relevant anticancer agent, after the initiation of therapy at the start of Cycle 1.

A starting dose of ibrutinib of 560 mg daily will be combined with the specified anticancer agent in the following tumor types, in 4 separate and parallel cohorts:

RCC: ibrutinib+everolimus

Urothelial carcinoma: ibrutinib+paclitaxel

Gastric adenocarcinoma: ibrutinib+docetaxel

CRC: ibrutinib+cetuximab

A dose level review committee (DLRC) will evaluate the safety data at the completion of the initial Phase 1b portion in each cohort to determine the RP2D, prior to continuing with enrollment into the Phase 2 portion.

A DLT is defined as any Grade 3 or higher non-hematologic or Grade 4 hematologic adverse event (AE) occurring during the DLT observation period (i.e., 21 days after the initiation of combination therapy at the start of Cycle 1) and considered to be at least possibly related to the study treatment with the following clarifications:
Grade 4 diarrhea and vomiting
Grade 3 nausea, diarrhea or vomiting despite maximum medical supportive care and persisting >3 days
Grade 3 fatigue persisting >7 days
Grade 3 infusion reaction that does NOT resolve with appropriate clinical management
Grade 3 rash lasting >7 days that does NOT resolve with appropriate clinical management
Grade 4 neutropenia for >7 days duration (irrespective of adequate growth factor support)
Grade 3 thrombocytopenia with clinically significant bleeding
Grade 4 thrombocytopenia

In the Phase 1b portion of the study, subjects who discontinue one or more study drugs, or require a dose reduction within 21 days after the initiation of therapy at the start of Cycle 1 will be replaced, unless the discontinuation is in association with a DLT. Subjects who miss one or more scheduled doses of either study drug within 21 days after the initiation of therapy at the start of Cycle 1 will continue. However, such a subject will not be evaluable for DLT assessment, and will be replaced for DLT assessment purposes.

After Cycle 1, all subjects will be treated until unacceptable toxicity or disease progression, whichever occurs first.

Tumor assessment by CT/MRI will occur every 6 weeks (2 cycles) and will be evaluated according to RECIST 1.1 guidelines.

After the RP2D has been defined for each cohort, enrollment in Phase 2 will commence.

### Inclusion Criteria:

### Disease Related

1. Histologically confirmed:

RCC (clear cell)

Urothelial carcinoma (transitional cell)

Gastric or GEJ adenocarcinoma

K-RAS or N-RAS wild-type EGFR expressing CRC

2. One or more measurable lesions per RECIST 1.1 criteria.
3. The following prior criteria should be followed:

Metastatic RCC: minimum of 1 and maximum of 4 prior regimens, one or more of which must have included a VEGF-TKI

Advanced (locally recurrent and/or metastatic) urothelial carcinoma: minimum of 1 and maximum of 2 prior regimens, one of which must be a cisplatin based regimen

Advanced (locally recurrent and or metastatic) gastric or GEJ adenocarcinoma: minimum of 1 and maximum of 3 prior regimens one of which must be a fluoropyrimidine based regimen

Metastatic CRC: minimum of 2 and maximum of 4 prior regimens, which must have included both an irinotecan and an oxaliplatin based regimen or unable to tolerate irinotecan chemotherapy

4. Each subject must be assessed by the investigator to be a suitable candidate for treatment with everolimus, docetaxel, paclitaxel or cetuximab, as appropriate according to their type of cancer.
5. Female subjects of childbearing potential must have a negative serum or urine pregnancy test within 3 days of the first dose of study drug. Female subjects who are of non-reproductive potential (ie, post-menopausal by history—no menses for ≥1 year; OR history of hysterectomy; OR history of bilateral tubal ligation; OR history of bilateral oophorectomy) are exempt from this criterion.
6. Male and female subjects of reproductive potential must agree to perform complete abstinence or to use both, a highly effective method of birth control (implants, injectables, combined oral contraceptives, some intrauterine devices [IUDs], or sterilized partner) and a barrier method (eg, condoms, cervical rings, cervical condoms, sponge) during the period of therapy and for 90 days after the last dose of ibrutinib, everolimus, docetaxel, and paclitaxel; 6 months after the last dose of cetuximab.(6 months for all study drugs UK only)

### Laboratory

7. Adequate hematologic function (independent of transfusion and growth factor support for at least 7 days prior to enrollment, with the exception of pegylated G-CSF (pegfilgrastim) and darbopoeitin which require at least 14 days prior to enrollment defined as:
Absolute neutrophil count≥1500 cells/mm³ (1.5×10⁹/L)
Platelet count>80,000 cells/mm³ (80×10⁹/L) for cohorts 1 (RCC)
Platelet counts>100,000 cells/mm³ (100×10⁹/L) for cohorts 2 (urothelial carcinoma) and 3 (gastric adenocarcinoma) and 4 (CRC)
Hemoglobin≥8.0 g/dL. for cohorts 1 (RCC), 2 (urothelial carcinoma), and 3 (gastric adenocarcinoma)
Hemoglobin≥9.0 g/dL for cohort 4 (CRC)
   8. Adequate hepatic and renal function defined as:
Serum aspartate transaminase (AST) and/or alanine transaminase (ALT)≤5.0× upper limit of normal (ULN) if liver metastases, or ≤3×ULN without liver metastases
Alkaline phosphatase<3.0×ULN or ≤5.0×ULN if liver or bone metastases present
Bilirubin≤1.5×ULN (unless bilirubin rise is due to Gilbert's syndrome or of non-hepatic origin, such as hemolysis) with the exception of patients in the gastric adenocarcinoma cohort where docetaxel is administered, these patients must have bilirubin within normal limits (WNL).
Estimated Creatinine Clearance≥30 mL/min (Cockcroft-Gault)

### Demographic

9. Men and women≥18 years of age
Eastern Cooperative Oncology Group (ECOG) performance status 0-1. For subjects with RCC or CRC, an ECOG score of 2, may be acceptable if approved by the medical monitor.

### Exclusion Criteria

### Disease-Related

1. Anticancer therapy (chemotherapy, antibody therapy, molecular targeted therapy, or investigational agent) within 28 days of the first dose of study drug (6 weeks for nitrosureas, mitomycin C, or antibody based therapies)
2. Prior treatment with:
Everolimus or temsirolimus (RCC cohort)
Any taxane (urothelial carcinoma cohort)
Any taxane (gastric adenocarcinoma cohort)
Cetuximab or panitumumab (CRC cohort)
   3. Prior radiotherapy to measurable lesion, unless documented progression has occurred post-irradiation
   4. Lack of recovery from previous therapeutic radiation (persistence of Grade≥2 radiation-related toxicity), or planned radiation therapy during the study period

### Concurrent Conditions

5. Any uncontrolled active systemic infection including any infection requiring systemic IV treatment which was completed ≤7 days before Cycle 1 Day 1.
6. History of other malignancies, except:
Malignancy treated with curative intent and with no known active disease present for ≥3 years before the first dose of study drug and felt to be at low risk for recurrence by investigator
Adequately treated non-melanoma skin cancer or lentigo maligna without evidence of disease
Adequately treated carcinoma in situ without current evidence of disease
   7. Prior treatment with ibrutinib or other BTK inhibitor
   8. ALT and/or AST>1.5×ULN and alkaline phosphatase>2.5×ULN (gastric adenocarcinoma cohort only)
   9. Known allergy or hypersensitivity to ibrutinib or any other component of combination therapy, including polysorbate 80 or Cremophor® EL (polyoxyethylated castor oil)
   10. Unresolved toxicities from prior anticancer therapy, defined as having not resolved to Common Terminology Criteria for Adverse Event (CTCAE, version 4.03), grade 0 or 1
   11. Known bleeding disorders (e.g., von Willebrand's disease) or hemophilia
   12. Grade≥3 sensory peripheral neuropathy
   13. History of stroke or intracranial hemorrhage within 6 months prior to enrollment
   14. Known brain or leptomeningeal disease (CT or MRI scan of the brain required only in case of clinical suspicion of central nervous system involvement)
   15. Known history of human immunodeficiency virus (HIV) or active with hepatitis C virus (HCV) or hepatitis B virus (HBV)
   Patients who are positive for hepatitis B core antibody, hepatitis B surface antigen or hepatitis C antibody must have a negative polymerase chain reaction (PCR) result before enrollment. Those who are PCR positive will be excluded.
   16. Major surgery within 4 weeks of first dose of study drug
   17. Any life-threatening illness, medical condition, or organ system dysfunction which, in the investigator's opinion, could compromise the subject's safety or put the study outcomes at undue risk
   18. Currently active, clinically significant cardiovascular disease, such as uncontrolled arrhythmia or Class 3 or 4 congestive heart failure, as defined by the New York Heart Association Functional Classification; or a history of myocardial infarction, unstable angina, or acute coronary syndrome within 6 months prior to enrollment
   19. Malabsorption syndrome, disease significantly affecting gastrointestinal function, or symptomatic inflammatory bowel disease or ulcerative colitis, or partial or complete bowel obstruction
   20. Unable to swallow capsules and/or tablets
   21. Concomitant use of warfarin or other Vitamin K antagonists
   22. Requires treatment with a strong cytochrome P450 (CYP) 3A4/5 inhibitor
   23. Lactating or pregnant
   24. Unwilling or unable to participate in all required study evaluations and procedures. Unable to understand the purpose and risks of the study and to provide a signed and dated informed consent form (ICF) and authorization to use protected health information (in accordance with national and local subject privacy regulations).

### Study Treatment

One cycle of treatment is 21 days in length and consists of daily administration of ibrutinib in combination with the relevant anticancer agent. Treatment will continue as long as the subject is without disease progression and not experiencing unacceptable toxicity.

TABLE 1

| Cohort | DL-1 | DL 1 | DL 2 |
|---|---|---|---|
| Cohort 1 | ibrutinib: 420 mg PO qd | ibrutinib: 560 mg PO qd | ibrutinib: 840 mg PO qd |
| RCC | everolimus: 10 mg PO qd | everolimus: 10 mg PO qd | everolimus: 10 mg PO qd |
| Cohort 2 | ibrutinib: 420 mg PO qd | ibrutinib: 560 mg PO qd | ibrutinib: 840 mg PO qd |
| Urothelial Carcinoma | paclitaxel: 80 mg/m² IV | paclitaxel: 80 mg/m² IV | paclitaxel: 80 mg/m² IV |
| | qweek | qweek | qweek |
| Cohort 3 | ibrutinib: 420 mg PO qd | ibrutinib: 560 mg PO qd | ibrutinib: 840 mg PO qd |
| Gastric Adenocarcinoma | docetaxel: 75 mg/m² IV | docetaxel: 75 mg/m² IV | docetaxel: 75 mg/m² IV |
| | q3weeks | q3weeks | q3weeks |
| Cohort 4 | ibrutinib: 420 mg PO qd | ibrutinib: 560 mg PO qd | ibrutinib: 840 mg PO qd |
| CRC | cetuximab: 400 mg/m² | cetuximab: 400 mg/m² | cetuximab: 400 mg/m² |
| | IV, then 250 mg/m² | IV, then 250 mg/m² | IV, then 250 mg/m² |
| | qweek | qweek | qweek |

PO = orally, qd = daily, qweek = weekly, q3weeks = every 3 weeks

TABLE 2

| | 769-P | 786-0 | A498 | ACHN | Caki-1 | RENCA |
|---|---|---|---|---|---|---|
| Type | clear cell | clear cell | clear cell | clear cell | clear cell | Mouse line |
| | adenocarcinoma | adenocarcinoma | adenocarcinoma | adenocarcinoma | adenocarcinoma | |
| Mutated | Homozygous | Homozygous | Heterozygous | No | No | |
| VHL | No HIF-1α/2α | No HIF-1α/2α, | No HIF-1α/2α, | | | |
| | | High HIF-2α | High HIF-2α | | | |
| Other | BAP1 | PTEN, MLH1, | SETD2 | NF2, PBRM1 | SSX1, SETD2 | |
| major | | NUP214 | | | | |
| mutations | | | | | | |

The examples and embodiments described herein are for illustrative purposes only and various modifications or changes suggested to persons skilled in the art are to be included within the spirit and purview of this application and scope of the appended claims.

1-37. (canceled)

38. A method for treating an urothelial carcinoma comprising co-administering to an individual in need thereof a therapeutically effective amount of a combination of a BTK inhibitor and paclitaxel.

39-130. (canceled)
